(19) United States
(12) Patent Application Publication
(10) Pub. No.: US 2024/0252200 A1
(43) Pub. Date: Aug. 1, 2024

(54) SKIN TREATMENT SYSTEMS AND METHODS

(71) Applicant: Cytrellis Biosystems, Inc., Woburn, MA (US)

(72) Inventors: Karen Cronholm, Arlington, MA (US); Stephen Gemmell, Andover, MA (US); Cathleen O. Alaimo, Indianapolis, IN (US); J. Christopher Flaherty, Nottingham, NH (US)

(21) Appl. No.: 18/560,784

(22) PCT Filed: May 20, 2022
(86) PCT No.: PCT/US22/30236

§ 371 Date: Nov. 14, 2023

Related U.S. Application Data
(60) Provisional application No. 63/190,904, filed on May 20, 2021.
(60) Provisional application No. 63/248,562, filed on Sep. 27, 2021.

(51) Int. Cl. A61B 17/3209; A61B 17/00

(52) U.S. Cl. CPC A61B 17/32093; A61B 2017/00761

(57) ABSTRACT

Systems for producing a cosmetic effect in skin tissue of a patient are provided. The systems comprise a treatment module and an actuation assembly. The treatment module comprises at least one coring element configured to remove a portion of skin tissue when the coring element is inserted into and withdrawn from the skin tissue. The actuation assembly is operably attached to the treatment module and is configured to translate and/or actuate the treatment module in one or more directions relative to a surface of the skin tissue. The system is configured to perform a microcoring procedure that provides a cosmetic effect to the patient. Methods of performing a microcoring procedure are also provided.

### RELATED APPLICATIONS

This application claims the benefit of U.S. Provisional Patent Application Ser. No. 63/190,904, titled “Skin Treatment Systems and Methods”, filed May 20, 2021, the content of which is incorporated herein by reference in its entirety for all purposes.

This application claims the benefit of U.S. Provisional Patent Application Ser. No. 63/248,562, titled “Skin Treatment Systems, Devices and Methods”, filed Sep. 27, 2021, the content of which is incorporated herein by reference in its entirety for all purposes.

This application is related to U.S. patent application Ser. No. 14/764,866, titled “Methods and Devices for Skin Tightening”, filed Jul. 30, 2015, U.S. Pat. No. 10,543,127, issued Jan. 18, 2020, the content of which is incorporated herein by reference in its entirety for all purposes.

This application is related to U.S. patent application Ser. No. 15/905,421, titled “Methods and Devices for Skin Tightening”, filed Feb. 26, 2018, U.S. Pat. No. 10,251,792, issued Apr. 9, 2019, the content of which is incorporated herein by reference in its entirety for all purposes.

This application is related to U.S. patent application Ser. No. 16/707,122, titled “Methods and Devices for Skin Tightening”, filed Dec. 9, 2019, Publication No. US 2020/0188184, published Jun. 18, 2020, the content of which is incorporated herein by reference in its entirety for all purposes.

This application is related to U.S. patent application Ser. No. 17/207,172, titled “Microclosures and Related Methods for Skin Treatment”, filed Mar. 19, 2021, United States Publication No. 2021/0322005, published Oct. 21, 2021, the content of which is incorporated herein by reference in its entirety for all purposes.

This application is related to U.S. patent application Ser. No. 14/910,767, titled “Methods and Apparatuses for Skin Treatment using Non-Thermal Tissue Ablation”, filed Feb. 8, 2016, U.S. Pat. No. 10,555,754, issued Feb. 11, 2020, the content of which is incorporated herein by reference in its entirety for all purposes.

This application is related to U.S. patent application Ser. No. 16/722,069, titled “Methods and Apparatuses for Skin Treatment using Non-Thermal Tissue Ablation”, filed Dec. 20, 2019, United States Publication No. 2020/0121354, published Apr. 23, 2020, the content of which is incorporated herein by reference in its entirety for all purposes.

This application is related to U.S. patent application Ser. No. 15/106,036, titled “Methods and Devices for Manipulating Subdermal Fat”, filed Jun. 17, 2016, U.S. Pat. No. 10,953,143, issued Mar. 23, 2021, the content of which is incorporated herein by reference in its entirety for all purposes.

This application is related to U.S. patent application Ser. No. 17/166,543, titled “Methods and Devices for Manipulating Subdermal Fat”, filed Feb. 3, 2021, United States Publication No. 2021/0178028, published Jun. 17, 2021, the content of which is incorporated herein by reference in its entirety for all purposes.

This application is related to U.S. patent application Ser. No. 15/526,299, titled “Devices and Methods for Ablation of the Skin”, filed May 11, 2017, U.S. Pat. No. 11,324,534, issued May 10, 2022, the content of which is incorporated herein by reference in its entirety for all purposes.

This application is related to U.S. patent application Ser. No. 17/709,542, titled “Devices and Methods for Ablation of the Skin”, filed Mar. 31, 2022, United States Publication No. ______, published______, the content of which is incorporated herein by reference in its entirety for all purposes.

This application is related to United States Design Patent Application Serial Number 29/509,219, titled “Device and Device Body for Mechanical Fractional Ablation of the Skin”, filed Nov. 14, 2014, United States Design Patent No. D797286, issued Sep. 12, 2017, the content of which is incorporated herein by reference in its entirety for all purposes.

This application is related to U.S. patent application Ser. No. 16/090,034, titled “Devices and Methods for Cosmetic Skin Resurfacing”, filed Sep. 28, 2018, U.S. Pat. No. 11,166,743, issued Nov. 9, 2021, the content of which is incorporated herein by reference in its entirety for all purposes.

This application is related to U.S. patent application Ser. No. 17/491,691, titled “Devices and Methods for Cosmetic Skin Resurfacing”, filed Oct. 1, 2021, United States Publication No. 2022-0125477, published Apr. 28, 2022, the content of which is incorporated herein by reference in its entirety for all purposes.

This application is related to U.S. patent application Ser. No. 16/335,028, titled “Devices and Methods for Cosmetic Skin Resurfacing”, filed Mar. 20, 2019, United States Publication No. 2019/0366067, published Dec. 5, 2019, the content of which is incorporated herein by reference in its entirety for all purposes.

This application is related to U.S. patent application Ser. No. 16/857,801, titled “Rapid Skin Treatment Using Microcoring”, filed Apr. 24, 2020, United States Publication No. 2020/0246039, published Aug. 6, 2020, the content of which is incorporated herein by reference in its entirety for all purposes.

This application is related to U.S. patent application Ser. No. 17/291,235, titled “Systems and Methods for Skin Treatment”, May 4, 2021, United States Publication No. 2021/0401453, published Dec. 30, 2021, the content of which is incorporated herein by reference in its entirety for all purposes.

### TECHNICAL FIELD

The embodiments disclosed herein relate generally to systems, devices, and methods for treatment of biological tissues.

### BACKGROUND

Many human health issues arise from damage, deterioration, or loss of tissue due to disease, advanced age, and/or injury. These health issues can manifest themselves in a variety of alterations of tissue structure and/or function, including scarring, sclerosis, tightness, and laxity. In aesthetic medicine, elimination of excess tissue and/or skin laxity is an important concern that affects more than 25% of the U.S. population.

### BRIEF SUMMARY

There is a need for improved systems and methods that provide increased effectiveness over currently available techniques while maintaining convenience, affordability, and accessibility to patients desiring tissue restoration.

According to an aspect of the present inventive concepts, a system for producing a cosmetic effect in skin tissue of a patient comprises: a treatment module comprising at least one coring element configured to remove a portion of skin tissue when the coring element is inserted into and withdrawn from the skin tissue; and an actuation assembly operably attached to the treatment module and configured to translate and/or actuate the treatment module in one or more directions relative to a surface of the skin tissue. The system can be configured to perform a microcoring procedure that provides a cosmetic effect to the patient.

In some embodiments, the system is configured to perform the microcoring procedure while avoiding heating of skin tissue.

In some embodiments, the system is configured to perform the microcoring procedure while avoiding cell necrosis due to thermal injury.

In some embodiments, the system is configured to perform the microcoring procedure and achieve expedited patient recovery.

In some embodiments, the system is configured to perform the microcoring procedure and achieve rapid wound closure. The rapid wound closure can include near immediate closure along relaxed skin tension lines of the patient. The rapid wound closure can comprise wound closure within one week of the performance of the microcoring procedure.

In some embodiments, the system is configured to perform the microcoring procedure and achieve resolution of erythema within two weeks of the performance of the microcoring procedure.

In some embodiments, the system is configured to perform the microcoring procedure and achieve minimal side effects to the patient.

In some embodiments, the system is configured to perform the microcoring procedure and achieve significant skin tightening.

In some embodiments, the system is configured to perform the microcoring procedure and achieve both tightening of the patient's skin as well as a reduction in wrinkles and/or folds of the patient's skin. The system can be configured to perform the microcoring procedure and further achieve minimal or no scarring. The minimal or no scarring can comprise scarring with a Manchester Scar Scale value of less than 10.

In some embodiments, the system is configured to perform the microcoring procedure and achieve minimal or no scarring. The minimal or no scarring can comprise scarring with a Manchester Scar Scale value of less than 10.

In some embodiments, the system is configured to perform the microcoring procedure and achieve a healing response as described in reference to FIG. 5.

In some embodiments, the system is configured to perform the microcoring procedure and achieve an increase in skin thickness. The increase in skin tissue thickness can comprise an increase in epidermal and/or papillary dermal thickness. The increase in skin thickness can comprise an increase as described in reference to FIG. 8. The system can be configured to perform the microcoring procedure and further achieve an increase in collagen content.

In some embodiments, the system is configured to perform the microcoring procedure and achieve an increase in collagen content. The increase in collagen content can comprise an increase of at least 30%, at least 50%, and/or at least 70%. The increase in collagen content can present three months after the performance of the microcoring procedure.

In some embodiments, the system is configured to perform the microcoring procedure and avoid the patient taking an antibiotic medication, antiviral medication, or both.

In some embodiments, the system is configured to perform the microcoring procedure and avoid inflicting the patient with significant levels of pain. The system can be configured to perform the microcoring procedure on the patient's abdomen, and to maintain pain levels for the patient during the microcoring procedure at a level at or below 5, 4, and/or 3 as measured on a scale of 0-10. The system can be configured to perform the microcoring procedure on the patient's face, and to maintain pain levels for the patient during the procedure at a level at or below 4, 3, 2, and/or 1 as measured on a scale of 0-10.

In some embodiments, the system is configured to perform the microcoring procedure and achieve at most moderate, trace, and/or no bleeding.

In some embodiments, the system is configured to perform the microcoring procedure and achieve a skin surface area reduction of at least 3%, 5%, and/or 7% of the area treated.

In some embodiments, the system is configured to perform the microcoring procedure and achieve a GAIS score of at least 1 and/or 2 at 90 days after the performance of the microcoring procedure.

In some embodiments, the system is configured to perform the microcoring procedure and achieve minimal change in skin pigmentation. The minimal change in skin pigmentation can comprise a minimal change in melanin index.

In some embodiments, the at least one coring element comprises one or more hollow needles. The one or more hollow needles can comprise three hollow needles. The three hollow needles can be in a linear arrangement.

In some embodiments, the system further comprises a spacer assembly constructed and arranged to stabilize and/or maintain a constant position of at least a portion of the system relative to the surface of the skin tissue.

In some embodiments, the system is configured to perform a microcoring procedure that achieves a therapeutic outcome comprising causing a physiologic effect selected from the group consisting of: adipose tissue remodeling and/or removal; dermal remodeling; dermal tightening; and combinations thereof.

In some embodiments, the system is configured to perform a microcoring procedure that achieves a therapeutic outcome comprising causing a physiologic improvement selected from the group consisting of: maintenance and/or remodeling of elastin; procollagen and/or collagen production; skin appearance, such as skin appearance that has been decreased by menopause; skin barrier repair and/or function; skin contour appearance; skin elasticity; skin luminosity; skin moisture; skin plumpness; skin softness; skin suppleness; skin tautness; skin texture and/or promotion of re-texturation; skin thickness; skin tone, radiance, and/or clarity; skin elasticity and/or resiliency; and combinations thereof.

In some embodiments, the system is configured to perform a microcoring procedure that achieves a therapeutic outcome comprising inhibiting the appearance of wrinkles.

In some embodiments, the system is configured to perform a microcoring procedure that achieves a therapeutic outcome comprising modification of a hair follicle.

In some embodiments, the system is configured to perform a microcoring procedure that achieves a therapeutic outcome comprising reducing a physiologic feature selected from the group consisting of: an acne scar; a cheek wrinkle; a dynamic wrinkle, fine wrinkle, and/or static wrinkle; an eye wrinkle; elastosis; a facial pore; a pigment spot; sebaceous gland activity; size of a wrinkle; a stretch mark; a surgical scar; a tattoo; and combinations thereof.

In some embodiments, the system is configured to perform a microcoring procedure that achieves a therapeutic outcome comprising reducing a tattoo.

In some embodiments, the system is configured to perform a microcoring procedure that achieves a therapeutic outcome comprising regeneration of skin.

In some embodiments, the system is configured to perform a microcoring procedure that achieves a therapeutic outcome comprising replenishing essential nutrients of the skin and/or replenishing constituents of the skin.

In some embodiments, the system is configured to perform a microcoring procedure that achieves a therapeutic outcome comprising restoring skin luster and/or skin brightness.

In some embodiments, the system is configured to perform a microcoring procedure that achieves a therapeutic outcome comprising treating and/or reducing a physiologic feature selected from the group consisting of: fine lines and/or wrinkles; one or more scars; skin sagging; and combinations thereof.

In some embodiments, the system further comprises an algorithm and one or more system parameters, and the algorithm is configured to adjust one or more system parameters in order to achieve a desired therapeutic outcome. The algorithm can be configured to automatically adjust the one or more system parameters. The algorithm can be configured to adjust the one or more system parameters based on one or more patient parameters. The system can further comprise one, two, or more sensors, and the one or more patient parameters can be recorded by the one, two, or more sensors.

In some embodiments, the system is configured to perform a microcoring procedure that achieves a therapeutic outcome comprising removal of skin cores without formation of scars.

In some embodiments, the system is configured to perform a microcoring procedure that achieves a therapeutic outcome comprising a pain score of no more than 3.5 and/or no more than 3.0.

In some embodiments, the system is configured to perform a microcoring procedure that achieves a therapeutic outcome comprising at least a 50%, 75%, and/or 85% likelihood of achieving an LWSS improvement of at least 0.1, 0.2, 0.3, and/or 0.5.

In some embodiments, the system is configured to perform a microcoring procedure that achieves a therapeutic outcome comprising at least a 50%, 75%, and/or 85% likelihood of achieving a GAIS improvement of at least 1.0.

In some embodiments, the system is configured to perform a microcoring procedure that achieves a therapeutic outcome comprising at least a 50%, 75%, and/or 85% likelihood of achieving a significant level of patient satisfaction.

In some embodiments, the system is configured to perform a microcoring procedure that achieves a therapeutic outcome comprising at least a 50%, 75%, and/or 85% likelihood of achieving no more than mild bleeding post-procedure.

In some embodiments, the system is configured to perform a microcoring procedure that achieves a therapeutic outcome comprising at least a 50%, 75%, and/or 85% likelihood of avoiding a significant response related to ecchymosis, edema, erythema, hyperpigmentation, itching, pain and/or discomfort, redness, tenderness, and/or tightness.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a block diagram of a system for treating and/or diagnosing tissue, consistent with the present inventive concepts.

FIG. 2 illustrates a side view of a coring element being introduced into the skin, consistent with the present inventive concepts.

FIG. 3 is a table of inclusion and exclusion criteria for various human clinical studies performed by applicant, consistent with the present inventive concepts.

FIG. 4 is a photograph of a treatment area immediately after microcore removal, consistent with the present inventive concepts.

FIG. 5 is a table of average score healing profiles of human patients of a clinical study performed by applicant, consistent with the present inventive concepts.

FIG. 6 is a series of photographs of treatment sites depicting wound healing after microcoring treatment, consistent with the present inventive concepts.

FIGS. 7A-B are photographs of histological slides prepared from biopsies of two patients that received a microcoring treatment, consistent with the present inventive concepts.

FIG. 8 is a table of skin thickness changes of human patients of a clinical study performed by applicant, consistent with the present inventive concepts.

FIG. 9 illustrates a top view and a magnified view of an abdomen of a human patient showing healing of microcores along RSTLs, consistent with the present inventive concepts.

FIGS. 10A-D illustrate a series of views of a coring element, consistent with the present inventive concepts.

FIG. 11 is a table of the baseline demographic variables for a human clinical study performed by applicant, consistent with the present inventive concepts.

FIG. 12 is a table of the mean post-procedure pain scores as reported by clinical study patients, consistent with the present inventive concepts.

FIG. 13 is a series of representative patient photographs taken before and after treatment, consistent with the present inventive concepts.

FIG. 14 is a graph demonstrating the GAIS change before and after treatment by side, consistent with the present inventive concepts.

FIG. 15 is a graph demonstrating patient satisfaction after treatment by side, consistent with the present inventive concepts.

FIG. 16 is a series of representative patient photographs taken after treatment, consistent with the present inventive concepts

### DETAILED DESCRIPTION OF THE DRAWINGS

Reference will now be made in detail to the present embodiments of the systems, devices, and methods (singly or collectively “technology” or “technologies” herein), examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

It will be understood that the words “comprising” (and any form of comprising, such as “comprise” and “comprises”), “having” (and any form of having, such as “have” and “has”), “including” (and any form of including, such as “includes” and “include”) and/or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of two or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, “prevention” and the like, where used herein, shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean or and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. “Positive pressure” includes pressure above room pressure or simply a pressure that is greater than another pressure, such as a positive differential pressure across a fluid pathway component such as a valve. “Negative pressure” includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can completely surround the component.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

As used herein, the term “conduit” or “conduits” can refer to an elongate component that can include one or more flexible and/or non-flexible filaments selected from the group consisting of: one, two or more wires or other electrical conductors (e.g. including an outer insulator); one, two or more wave guides; one, two or more hollow tubes, such as hydraulic, pneumatic, and/or other fluid delivery tubes; one or more optical fibers; one, two or more control cables and/or other mechanical linkages; one, two or more flex circuits; and combinations of these. A conduit can include a tube including multiple conduits positioned within the tube. A conduit can be configured to electrically, fluidically, sonically, optically, mechanically, and/or otherwise operably connect one component to another component.

As used herein, the term “transducer” is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as: light (e.g. a transducer comprising a light emitting diode or light bulb); sound (e.g. a transducer comprising one or more piezoelectric and/or CMUT transducers configured to deliver and/or receive ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: heat energy to tissue; cryogenic energy to tissue; electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising one or more piezoelectric and/or CMUT transducers); chemical energy; electromagnetic energy; magnetic energy; and combinations of two or more of these. Alternatively or additionally, a transducer can comprise a mechanism, such as: a valve; a grasping element; an anchoring mechanism; an electrically-activated mechanism; a mechanically-activated mechanism; and/or a thermally activated mechanism.

As used herein, the term “functional element” is to be taken to include one or more elements constructed and arranged to perform a function. A functional element can comprise one or more sensors and/or one or more transducers. In some embodiments, a functional element is configured to deliver energy to tissue, such as to treat and/or image tissue. In some embodiments, a functional element comprises one or more hollow filaments (e.g. one or more needles) that are configured to be inserted into tissue and/or withdrawn from tissue, such as to perform a microcoring treatment as described herein. In some embodiments, a functional element (e.g. comprising one or more sensors) can be configured to record one or more parameters, such as a patient physiologic parameter; a patient anatomical parameter (e.g. a tissue parameter); a patient environment parameter; and/or a system parameter (e.g. temperature and/or pressure within the system). In some embodiments, a sensor or other functional element is configured to perform a diagnostic function (e.g. to gather data used to perform a diagnosis). In some embodiments, a functional element is configured to perform a therapeutic function (e.g. perform a microcoring procedure, deliver therapeutic energy, and/or deliver a therapeutic agent). In some embodiments, a functional element comprises one or more elements constructed and arranged to perform a function selected from the group consisting of: core and/or remove tissue; deliver energy; extract energy (e.g. to cool a component); deliver a drug or other agent; manipulate a system component or patient tissue; record or otherwise sense a parameter such as a patient physiologic parameter or a patient anatomical parameter; and combinations of two or more of these. A “functional assembly” can comprise an assembly constructed and arranged to perform a function, such as are described hereabove. In some embodiments, a functional assembly is configured to core tissue and/or otherwise treat tissue (e.g. a functional assembly configured as a treatment assembly or treatment module). Alternatively or additionally, a functional assembly can be configured as a diagnostic assembly that records one or more parameters, such as a patient physiologic parameter; a patient anatomical parameter; a patient environment parameter; and/or a system parameter. A functional assembly can comprise a deployable assembly, such as a robotically controlled assembly. A functional assembly can comprise one or more functional elements.

As used herein, the term “agent” shall include but not be limited to one or more agents selected from the group consisting of: an agent configured to improve and/or maintain the health of a patient; a drug (e.g. a pharmaceutical drug); a hormone; a protein; a protein derivative; a small molecule; an antibody; an antibody derivative; an excipient; a reagent; a buffer; a vitamin; a nutraceutical; and combinations of these.

As used herein, the term “target tissue” comprises one or more volumes of tissue of a patient to be diagnosed and/or treated. Similarly, a “treatment target” or “tissue target” comprises one or more volumes of tissue to be diagnosed and/or treated. “Safety margin tissue” comprises tissue whose treatment (e.g. receiving of a microcoring treatment) yields no significant adverse effect to the patient. “Non-target tissue” comprises tissue that is not intended to receive treatment (e.g. not intended to receive a microcoring treatment).

As used herein, the term “system parameter” comprises one or more parameters of the system of the present inventive concepts. A system parameter can comprise one or more “tissue treatment parameters” (also referred to as “tissue treatment settings”), such as one, two or more tissue treatment parameters selected from the group consisting of: a “microcoring parameter” (also referred to as a “coring parameter” herein); a target level of a patient parameter such as a patient diagnostic parameter and/or a patient environment parameter as described herein; a tissue-type parameter; a tissue target area parameter; a tissue anatomical location area parameter; and combinations of these. Microcoring parameters include but are not limited to: depth of penetration of a coring element; duration and/or speed of penetration of a coring element such as rise time of speed of penetration of a coring element; penetration dwell time (also referred to as “hold time”); duration and/or speed of withdrawal of a coring element; time between penetrations; density of coring (also referred to as “microcoring density”); spacing between coring elements; coring diameter; location of penetration; coring suction force; skin suction force (e.g. vacuum pressure and contact area); vacuum “pinch” time (e.g. time to release skin suction); vacuum regeneration time (e.g. as dictated by tubing and/or filter volume and controlled leaks in the system); frequency of coring; inner diameter surface friction of coring element; and combinations of these. A system parameter can comprise a parameter selected from the group consisting of: a tissue treatment parameter; a microcoring parameter; an energy delivery parameter; a pressure level; a temperature level; an energy level; a power level; a frequency level; an amplitude level; a battery level; a threshold level for an alarm or other alert condition; and combinations of these. A system parameter can include one or more tissue targets identified to be treated (e.g. areas of skin tissue to be treated), such as tissue targets identified for treatment by an operator and/or by an algorithm of the system.

As used herein, the term “patient parameter” comprises one or more parameters associated with the patient. A patient parameter can comprise a patient physiologic parameter, such as a physiologic parameter selected from the group consisting of: temperature (e.g. tissue temperature); pressure such as blood pressure or other body fluid pressure; pH; a blood gas parameter; blood glucose level; hormone level; heart rate; respiration rate; and combinations of these. Alternatively or additionally, a patient parameter can comprise a patient environment parameter, such as an environment parameter selected from the group consisting of: patient geographic location; temperature; pressure; humidity level; light level; time of day; and combinations of these.

As used herein, the term “image data” comprises data created by one or more imaging devices. Image data can include data related to target tissue, safety margin tissue, and non-target tissue. Image data can also include data related to any implants or other non-tissue objects that are proximate tissue being imaged. Image data can be processed by one or more algorithms of the present inventive concepts, such as to determine one or more locations to treat (e.g. target tissue identified to be ablated or otherwise receive microcoring or other treatment), and/or to determine one or more locations to which treatment (e.g. microcoring) is to be avoided (e.g. non-target tissue). Image data can comprise data produced by a single imaging component, or from multiple imaging components.

As used herein, the term “transmitting a signal” and its derivatives shall refer to the transmission of power and/or data between two or more components, in any direction, such as via wired or wireless connections.

As used herein, the term “patient use data” shall refer to data related to use of the tissue treatment systems of the present inventive concepts on a patient (e.g. use of the system in a diagnostic and/or therapeutic procedure performed on a patient). The data can include but is not limited to: operating parameters such as tissue treatment parameters; target tissue parameters such as location of target tissue and/or amount of target tissue to be treated; patient parameters such as patient physiologic parameters and/or patient location or other patient environment parameters; clinician parameters; clinical site parameters; and combinations of these. Patient use data can include data from multiple patients, such as data collected from multiple patients that interface with (e.g. receive a treatment from) one or more systems of the present inventive concepts. In some embodiments, an algorithm of the present inventive concepts uses patient use data from one or more patients to determine a system parameter to be used in performing a medical procedure on a patient.

The systems, devices, and method of the present inventive concepts can be configured for treating skin (e.g., eliminating tissue volume, tightening skin, lifting skin, reducing skin laxity, and/or otherwise providing a cosmetic effect), such as by selectively excising a plurality of microcores of patient tissue. In some embodiments, the tissue is treated without thermal energy being imparted to surrounding (e.g., non-excised) tissue. These systems, devices, and methods satisfy an unmet need for rapid and safe treatment of skin (“skin” or “skin tissue” herein), including, for example, faster pretreatment preparation and post-treatment healing times as compared to current surgical and thermal treatment methods.

### Microcoring

In general, the term “microcoring,” as used herein, refers to technologies that utilize one or more (in some embodiments, a plurality, e.g., an array) hollow needles, or other non-thermal treatment elements (e.g., blades, tubes, and/or drills) that remove and/or otherwise treat tissue of a patient. These treatment elements can be of sufficiently small dimension (e.g. comprise a sufficiently small diameter) to minimize the extent of bleeding and/or clotting within holes or slits, and/or to minimize scar formation, when used to excise (e.g. and optionally sequester) tissue from a site. In some embodiments, excising a tissue means forming a tissue portion (e.g. a “microcore”), such as by inserting a hollow needle into the site so that the tissue portion is formed inside the hollow needle and severed from surrounding tissue, whereby a microcore that is separated (e.g. physically separated) from other tissue is generated.

In some embodiments, microcoring elements, assemblies, and/or other components as described herein may include a component configured to perform sequestration of the excised tissue. As used herein, the term “sequestering”, when used in reference to tissue, means excising a microcore and then removing the excised microcore from the excision site. In certain embodiments, sequestered tissue may be permanently disposed. In certain embodiments, sequestered tissue may be used for diagnostic purpose, such as when used for biopsy and/or histology analyses, such as those known in the art. In some embodiments, technologies provided herein maximize removal and/or minimize risk of (partial or complete) re-insertion of extracted tissue.

It should be understood that particular microcoring technologies using hollow needles specifically described herein serve for exemplary and/or illustrative purposes, and that other techniques and devices can be used to create microcores. Microcoring technologies described herein may include a number of advantageous features. For example, provided technologies may enable visualization of results in real time during the course of the treatment, such as through feedback (e.g. patient and/or clinician feedback) and subsequent treatment adjustment in real time.

Alternatively or additionally, the systems and devices of the present inventive concepts that are used for microcoring can include micro-sized features that may be beneficial for controlling extent of skin treatment and/or minimize adverse effects of the skin treatment.

Still further, in some embodiments, technologies described herein may require less skill than that of a surgeon. Thus, in certain embodiments, a patient may be treated by a non-physician professional and/or in an outpatient setting, rather than in an inpatient, surgical setting. In some embodiments, a patient may be treated at a spa, at a cosmetic salon, or at home. That is, the technologies of the present inventive concepts are amenable to and/or permit consistent and/or reproducible administration of skin treatment procedures in a variety of treatment settings, and with a broad range of clinicians, technicians, and/or other operators (“operators” or “users” herein) performing the procedures.

In some embodiments, the technologies described herein may have generally a lower risk profile and/or the technologies can provide more predictable results and/or risk factors than those for more invasive techniques (e.g., plastic surgery) or energy-based techniques (e.g., laser, radiofrequency (RF), or ultrasound), which may or may not be invasive.

In some embodiments, non-thermal fractional excision technologies described herein allow skin tightening, skin lifting, and/or reduction of skin laxity without (or with significant reduction of) one or more common side effects of thermal treatment methods (e.g. thermal ablation and/or other treatment methods that increase and/or otherwise modify the temperature of tissue in order to provide a treatment to that tissue). Thermal ablation techniques prevent and/or inhibit skin tightening by allowing coagulation of tissue and formation of rigid tissue cores that cannot be compressed. Thermal ablation techniques create a three-dimensional heat-affected zone (HAZ) surrounding an immediate treatment site. While fractional ablative lasers may be used on or near heat-sensitive sites (e.g., eyes, nerves), for example when the laser does not penetrate more than 1 mm into the skin (resulting in a comparatively small HAZ), other thermal ablation techniques (e.g., ultrasound-based techniques and radiofrequency-based techniques) cannot be used in the vicinity of heat-sensitive sites because the HAZ may extend to heat sensitive tissues potentially causing undesired damage (e.g. permanent undesired damage). As will be appreciated by those skilled in the art, a “heat-sensitive site” is a site where exposure to radiation and/or elevated temperature is associated with a relatively high risk of unacceptable cosmetic and/or physiologic outcomes. In any event, technologies of the present inventive concepts described herein have generally a lower risk profile than, for example, thermal methods, at least in part due to a zone of tissue injury that is smaller than the zone of injury (e.g., the HAZ) of thermal methods.

In some embodiments, advantages of certain technologies described herein include a therapeutic benefit selected from the group consisting of: a particularly low (e.g. lesser than that observed with other techniques such as invasive techniques and/or thermal techniques) degree of erythema; faster resolution of erythema; lower percent incidence, severity, and/or term of skin discoloration (hyperpigmentation or hypopigmentation); low swelling and/or inflammation (e.g. as compared, with that observed with laser treatment and/or with ultrasound-based treatment); and combinations of these.

In some embodiments, the technologies provided herein can allow for rapid closing of holes and/or slits after excising tissue (e.g. within a few seconds after treating skin, such as within ten seconds), thereby minimizing extent of bleeding and/or clotting within holes and/or slits, and/or minimizing the extent of scar formation.

In some embodiments, the technologies provided herein may be useful for maximizing treatment effect while minimizing treatment time, such as by using rapid-fire reciprocating needles or needle arrays, and/or by using large needle arrays that allow for simultaneous excision of tens, hundreds, or even thousands of microcores.

In some embodiments, the technologies described herein may be useful for maximizing tightening effect while minimizing healing time and/or minimizing the time in which a cosmetic effect occurs, such as by optimizing tightening (e.g. by controlling the extent of skin pleating, such as by increasing the extent of skin pleating for some applications or skin regions and/or by decreasing the extent of skin pleating for other applications or skin regions, as described herein).

In some embodiments, the technologies described herein may provide efficient clearance of sequestered and/or partially ablated tissue, and/or provide efficient clearance of debris from ablated tissue portions, thus reducing time for healing and/or improving the skin tightening treatment (e.g. relative to laser-based and/or other thermal technologies).

In some embodiments, the technologies described herein may be configured to allow for efficient and effective positioning of skin prior to, during, and/or after tissue excision (e.g. excision including tissue sequestration). Positioning the skin can be critical to control skin-tightening direction, and it can ensure treatment occurs in the desired location and desired dimensions (e.g. thickness, width in a preferred direction, such as along or orthogonal to Langer lines).

Among other things, the systems, devices, and methods of the present inventive concepts can include microcoring technologies that are configured to achieve desirable (e.g. reduced) procedure times and/or can significantly improve one or more aspects of healing from a tissue treatment procedure (e.g. a tissue removal procedure), such as when compared to thermal methods.

### Systems and Components for Microcoring

Described herein are technologies, methods, and/or devices for treating skin, such as by selectively microcoring skin tissue. In particular, described herein are hollow needles or other hollow filaments (“needles” herein), as well as related systems (e.g. including kits), devices, and methods, capable of microcoring tissue portions by capturing and retaining the tissue portions inside a lumen of one or more hollow needles after insertion into and withdrawal from the skin. Microcored tissue portions can be removed from a lumen of a hollow needle and discarded. The process can be repeated to generate multiple microcored (also referred to as “cored” herein) skin tissue portions, in particular over a desired area of skin and located at chosen sites of the body of a patient. The hollow needles, kits, devices, methods, and other technologies described herein may provide increased effectiveness over currently available apparatuses and techniques while maintaining convenience, affordability, and accessibility to patients desiring tissue restoration.

In some embodiments, technologies described herein include a treatment device, such as a handheld treatment device. An example treatment device may include a treatment module (e.g. a needle hub) comprising at least one hollow needle configured to remove a portion of the skin tissue (e.g. a microcore) when the hollow needle is inserted into and withdrawn from the skin tissue. In some embodiments, a treatment device may include an activation assembly (e.g. a translation and/or actuation assembly) connected to the treatment module, such as to translate (e.g. along one, two, and/or three axes) and/or actuate the treatment module in one or more directions relative to a surface of the skin tissue. In some embodiments, a treatment device may include a spacer to stabilize and/or maintain a constant position of the treatment device relative to the surface of the patient's skin tissue. In some embodiments, a treatment device may include a hand piece including a hand piece shell, such as a housing that at least partially encases the activation assembly. In some embodiments, a hand piece and/or hand piece shell may include or may be connected to a spacer, such as a connection at a distal end of a treatment device (e.g., an end of a treatment device for contacting skin).

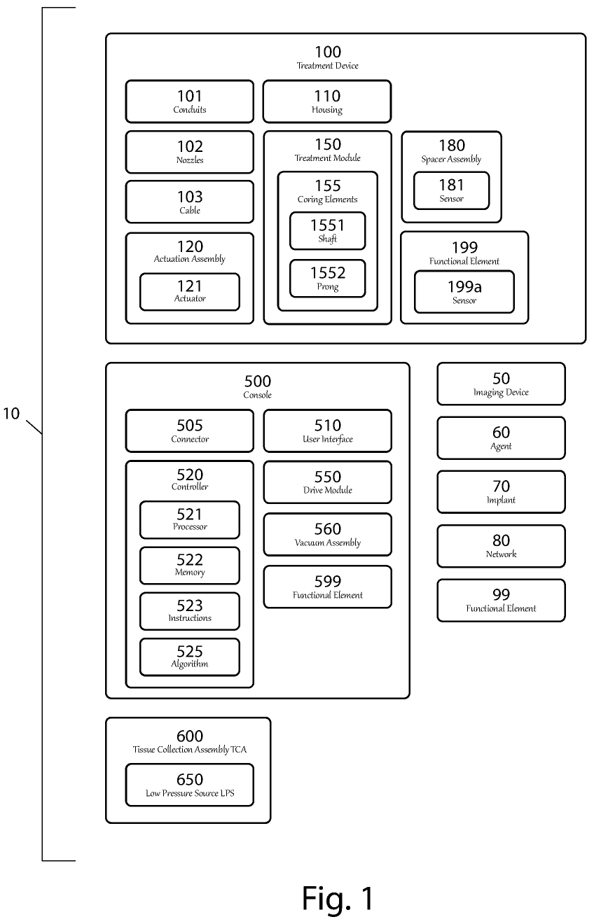

Referring now to FIG. 1, a schematic view of a tissue treatment system is illustrated, consistent with the present inventive concepts. System 10 can be configured to perform a medical procedure on a patient. A medical procedure performed using system 10 can include the performance of one or more clinical procedures, such as one or more diagnostic procedures and/or one or more treatment procedures (e.g. a tissue treatment procedure) performed on a patient. In some embodiments, system 10 is used by an operator (e.g. a clinician, technician, and/or other operator) to perform one, two or more clinical procedures, that are performed within a single day or over multiple days. System 10 can be configured to diagnose and/or treat one or more medical conditions (e.g. diseases, disorders, and/or cosmetic issues) of the patient. System 10 can be configured to treat and/or diagnose one or more portions (e.g. volumes) of patient tissue, “target tissue” herein. In some embodiments, system 10 comprises one, two or more devices that are configured to treat target tissue, such as to improve cosmesis of the patient (e.g. via microcoring as described herein). In some embodiments, system 10 is of similar construction and arrangement, and can include similar components, to the systems described in applicant's co-pending PCT application Serial Number PCT/US2019/060131, titled “Systems and Methods for Skin Treatment”, filed Nov. 6, 2019 [Docket Number 2012312-0261, CYT-010-PCT].

System 10 can include one or more devices that are configured to gather patient data, patient data PD herein. For example, system 10 can include one or more devices configured to collect patient data PD comprising patient diagnostic data, diagnostic data DD. Diagnostic data DD can comprise data related to a physiologic parameter of the patient, data related to the anatomy of the patient, data related to the environment of the patient (e.g. the current environment of the patient), and/or other patient-related data. Alternatively or additionally, system 10 can include one or more devices configured to collect patient image data, image data ID, which can comprise image data of tissue and/or one or more objects proximate tissue. Patient data PD can include data that is used in determining (e.g. by system 10 and/or an operator of system 10) a diagnosis and/or prognosis (either or both “diagnosis” herein) for the patient. Alternatively or additionally, patient data PD can include patient data that is used in a tissue treatment procedure (e.g. by system 10 and/or an operator of system 10), such as to guide or otherwise affect a microcoring and/or other treatment performed on the patient. Image data ID can include image data related to: target tissue; safety margin tissue; non-target tissue; an implanted diagnostic and/or a treatment device; a foreign body (e.g. a splinter, tattoo, and the like); and combinations of these. System 10 can be configured to produce image data ID through the delivery of energy, such as X-ray energy, sound energy (e.g. ultrasound energy), and/or light energy that is delivered and whose reflections and/or other transmissions are collected in order to produce image data ID. In some embodiments, image data ID comprises data related to tissue comprising blood, such as when image data ID comprises blood flow data (e.g. as obtained using Doppler ultrasound).

As used herein, a “tissue diagnostic procedure”, a “tissue diagnostic”, and their derivatives include but are not limited to: collection of diagnostic data DD; collection of image data ID (e.g. when system 10 records reflections and/or other transmissions of delivered X-ray, ultrasound, light, and/or other energy, and converts these recordings into image data ID); delivery of energy to tissue to characterize the tissue (e.g. when system 10 records one or more effects on the tissue due to the energy delivery, such as using spectroscopy); and/or recording of one or more tissue properties using one or more sensors and/or imaging devices of system 10. A tissue diagnostic procedure can also include a procedure in which various patient parameters are collected, such as patient environment parameters and/or a patient physiologic parameter, for example as described herein.

As used herein, a “tissue treatment procedure”, a “tissue treatment”, and their derivatives include but are not limited to: microcoring of tissue; removal of tissue; ablation of tissue; causing the necrosis of tissue; reducing the volume of tissue (e.g. debulking tissue); stimulating tissue; improving the strength of tissue (e.g. muscle tissue); manipulating and/or otherwise applying a force to tissue; stiffening tissue; and/or otherwise providing a cosmetic enhancement and/or other therapeutic effect to tissue.

System 10 includes treatment device 100 which can comprise one, two or more treatment devices that are configured to perform a treatment procedure on a patient (e.g. a microcoring or other tissue treatment procedure). Treatment device 100 can be configured to treat target tissue (e.g. perform a microcoring of target tissue). Alternatively or additionally, treatment device 100 can be configured to diagnose target tissue (e.g. gather diagnostic data DD and/or image data ID). Treatment device 100 can include one or more modules, treatment module 150 shown, each of can be configured to perform a patient treatment (e.g. a microcoring treatment). Treatment module 150 can comprise one, two, three or more filaments for coring tissue, coring elements 155 shown. Treatment device 100 can include actuation assembly 120 shown, which can comprise one, two or more assemblies configured to interface with treatment module 150, such as is described herein. Treatment device 100 can include spacer assembly 180 shown, which can comprise one or more assemblies that are constructed and arranged to be positioned between a corresponding one or more treatment modules 150 and tissue.

System 10 can include console 500 shown, which can comprise one, two or more discrete devices, where each of which can operably attach to one, two or more treatment devices 100, simultaneously and/or sequentially. Console 500 can include a connector, connector 505 as shown, which can be configured to operably attach (e.g. electrically, mechanically, fluidly, optically, sonically, and/or otherwise operably attach) to treatment device 100, such as via cable 103 of treatment device 100. Console 500 can be configured to allow an operator to control one or more treatment devices, such as via user interface 510 shown. Console 500 can comprise various assemblies and other components, as described herein, which singly or in combination are configured to provide to treatment device 100 one or more of: energy; mechanical, hydraulic, and/or pneumatic linkages; an agent (e.g. agent 60 described herein); and/or control signals. Console 500 can be configured to receive data from treatment device 100. In some embodiments, all or a portion of a console 500 is integrated into a treatment device 100 (e.g. the treatment device 100 is a relatively stand-alone device). Console 500 can comprise one or more algorithms, algorithm 525 shown. In some embodiments, treatment device 100 and/or another component of system 10 comprises all or a portion of algorithm 525.

System 10 can include imaging device 50 shown, which can comprise one, two or more imaging devices. Imaging device 50 can be configured to collect image data ID. In some embodiments, imaging device 50 comprises one, two or more imaging devices selected from the group consisting of: a fluoroscope or other X-ray imaging device; an ultrasound imager; a CT scanner; an MRI; an OCT imaging device; a camera such as a visual light camera and/or an infrared camera; and combinations of these. Imaging device 50 can comprise a device configured to characterize and/or otherwise collect data related to one or more properties of tissue, such as a device (e.g. an ultrasound-based device) configured to measure elasticity of tissue and/or other tissue property (e.g. with or without collecting an image of the tissue). In some embodiments, image data ID provided by imaging device 50 can be used to determine a target area to treat with system 10, and/or a non-target area to which treatment should be avoided. For example, algorithm 525 can be configured to analyze image data ID and provide feedback (e.g. suggestions and/or requirements) for particular tissue areas to be classified as target areas and/or non-target areas. In some embodiments, algorithm 525 is configured to identify one or more implants or other objects present under the patient's skin, to which treatment should be adjusted (e.g. avoided), such as an under-the-skin object comprising: a medical implant (e.g. implant 70 described herein) such as a cosmetic implant; a splinter; and/or tattoo ink. In these embodiments, algorithm 525 can be configured to identify a periphery of the under-the-skin object, such as to define a non-target zone including at least the area within the periphery (e.g. and also including a safety margin outside of the periphery).

System 10 can include agent 60 shown, which can comprise one or more pharmaceuticals and/or other agents that can be delivered to the patient. Agent 60 can comprise an agent that is applied topically and/or an agent that is delivered systemically (e.g. orally). Agent 60 can comprise one, two, or more agents selected from the group consisting of: hyaluronic acid; a moisturizer; an analgesic; a peptide; platelet rich plasma (PRP); arnica montana extract; a vasoconstrictor; methotrexate; minoxidil; stem cells; botulinum toxin; a corticosteroid; and combinations of these. Agent 60 can comprise an agent that is applied topically, and or inserted into the patient, such as into the dermis of the patient, such as when deposited in or otherwise proximate one or more target areas to be treated (e.g. pre-microcoring), during treatment (e.g. when deposited via coring elements 155 or otherwise), and/or after treatment (e.g. after microcoring). In some embodiments, functional element 99 comprises a delivery device configured to deliver agent 60, such as a syringe, needle, transdermal patch, microfluidic pump, and/or other delivery device configured to deliver agent 60 to the surface of the skin and/or to an internal location (e.g. into the dermis).

System 10 can include implant 70 shown, which can comprise one or more implants which can be implanted in the patient such as to improve cosmesis of the patient, and/or to treat a disease and/or disorder of the patient. In some embodiments, a treatment performed by system 10 includes the implantation of one or more implants 70, such as to further improve cosmesis of the patient. In some embodiments, a treatment performed by system 10 is adjusted due to the presence of an existing implant (e.g. implant 70), and/or due to a future implantation of an implant (e.g. implant 70).

System 10 can include tissue collection assembly 600 shown (also referred to as “TCA 600” herein), which can comprise one or more assemblies configured to collect tissue which has been removed from the patient by treatment module 150. TCA 600 can comprise one or more containers for storing collected tissue. TCA 600 can comprise a vacuum pump and/or other low-pressure source, LPS 650 shown, such as to create a pressure differential which causes tissue extracted by treatment device 100 to be drawn into TCA 600.

System 10 can include one or more functional elements, such as functional element 199 of treatment device 100, and/or functional element 599 of console 500, and/or functional element 99, each as shown. Functional elements 99, 199, and/or 599 can comprise one or more sensors and/or transducers, and/or an assembly that includes one or more sensors and/or transducers. Functional element 99, 199, and/or 599 can comprise a component (e.g. a sensor, or an assembly including a sensor) that is configured to collect patient data PD, such as diagnostic data DD and/or image data ID as described herein. In some embodiments, functional element 199 comprises at least one sensor, sensor 199a shown.

Functional elements 99, 199, and/or 599 can comprise one, two or more sensors configured to collect diagnostic data DD of a patient, and/or image data ID of a patient.

Functional element 99, 199, and/or 599 can comprise a wireless element, such as a wireless transmitter that can send and/or receive power and/or data wirelessly. In some embodiments, a functional element 99, 199, and/or 599 comprises a sensor and/or a transducer that receives power wirelessly, and/or transmits signals (e.g. recorded sensor signals) wirelessly.

Functional element 99, 199, and/or 599 can comprise one or more sensors selected from the group consisting of: accelerometer; gravity-based sensor; strain gauge; acoustic sensor (e.g. a microphone or other acoustic sensor); electromagnetic sensor (e.g. a hall effect sensor); pressure sensor; vibration sensor; temperature sensor; vacuum sensor; GPS sensor; pH sensor; optical sensor; and combinations of these.

Functional elements 99, 199, and/or 599 can comprise a patient “physiologic sensor” comprising one, two or more sensors configured to measure a patient physiologic parameter such as: body temperature; heart rate; blood pressure; respiration rate; perspiration rate; blood gas level; blood glucose level; brain and/or other neural activity such as measured by electroencephalogram (EEG), local field potential (LFP), and/or neuronal firing (e.g. single neuron firing activity); eye motion; EKG; and combinations of these.

Functional elements 99, 199, and/or 599 can comprise a patient “environment sensor” comprising one, two or more sensors configured to measure a patient “environment parameter” such as: room temperature; room humidity; room pressure; room light level; room ambient noise level; room barometric pressure; and combinations of these.

In some embodiments, functional elements 99, 199, and/or 599 comprise one or more sensors configured to measure a system 10 parameter, such as a system parameter selected from the group consisting of: temperature of at least a portion of a system 10 component; pressure and/or strain of a system 10 component; speed and/or acceleration of a system 10 component (e.g. speed and/or acceleration of a coring element 155 and/or other portion of treatment device 100); position and/or geometry of a system 10 component (e.g. position and/or geometry of a coring element 155 and/or other portion of treatment device 100); energy level; power level; and combinations of these.

In some embodiments, system 10 is configured to operate in a closed loop mode, in which one or more parameters of treatment device 100 are adjusted based on one or more recorded parameters, such as system parameters, patient physiologic parameters, and/or patient environment parameters, each as described herein. For example, algorithm 525 can analyze (e.g. continuously and/or intermittently analyze) one or more signals provided by a functional element 99, 199, and/or 599, and adjust the treatment performed by system 10 based on the analysis.

In some embodiments, functional elements 99, 199, and/or 599 comprise one or more transducers selected from the group consisting of: cooling element such as a Peltier element; heating element such as a Peltier element or a heat pump; vibrational transducer; light-producing element; a magnetic field-generating element; vacuum-generating element; and combinations of these.

In some embodiments, functional elements 99, 199, and/or 599 comprise an assembly or other component configured to provide a vacuum to another component of system 10. For example, functional elements 99, 199, and/or 599 can comprise a tissue-engaging port configured to receive a vacuum (e.g. from console 500) and to stabilize tissue, capture tissue (e.g. draw tissue toward the port) and/or otherwise engage tissue, when the vacuum is applied to the port. Functional elements 99, 199, and/or 599 can comprise a source of vacuum, such as vacuum that can be applied to such a tissue-engaging port.

In some embodiments, functional elements 99, 199, and/or 599 comprise an adhesive, and/or an adhesive dispensing component, such as when an adhesive is used to temporarily (e.g. less than 1 day) and/or chronically (e.g. at least 1 week, 1 month, or 3 months) attach a component of system 10 to tissue of the patient, and/or to another component of system 10.

In some embodiments, functional elements 99, 199, and/or 599 comprise a cooling fluid or cooling component (e.g. a thermoelectric cooling element) and/or an assembly configured to provide cooling (e.g. provide cooling to a system 10 component). In some embodiments, system 10 is configured to provide cooling to tissue and/or to a system 10 component during delivery of a tissue treatment and/or diagnosis, such as to avoid damage to non-target tissue and/or to avoid degradation of a system 10 component. Alternatively or additionally, system 10 can comprise a functional element comprising an assembly configured to provide a cooling fluid (e.g. in a recirculating arrangement) to another system 10 component.

In some embodiments, functional elements 99, 199, and/or 599 comprise an assembly or other component configured to apply a force to tissue (e.g. a grasping component configured to place tissue in tension, and/or a pushing element configured to provide a compressive force to tissue), such as to apply a force (e.g. a tensioning and/or compressing force) to tissue (e.g. target tissue) while a microcoring procedure is being performed on target tissue by another component of system 10.

Functional element 99, 199, and/or 599 can comprise an assembly configured to deliver agent 60 to the patient, as described herein. In some embodiments, agent 60 is delivered to the patient via one or more coring elements 155, where functional element 99, 199, and/or 599 comprises a pump or other fluid propulsion assembly that propels agent 60 through one or more conduits (e.g. fluid delivery tubes) such that agent 60 can be delivered into the patient (e.g. into the dermis of the patient) by one or more (e.g. all) coring elements 155 during a microcoring or other procedure performed via injection of elements 155 into the patient.

Functional element 99 can comprise a cell phone, laptop, tablet, camera, and/or other operator-maintained device. In some embodiments, data collected during a treatment procedure performed by system 10 is provided by, stored, and/or analyzed by one of these devices.

Functional element 99 can comprise a patient diagnostic device, such as a device configured to gather patient data PD (e.g. diagnostic data DD and/or image data ID).

Treatment device 100 comprises various components such as conduits 101, nozzles 102, cable 103, and housing 110. These components can be of similar construction and arrangement to the similar components described in applicant's co-pending PCT application Serial Number PCT/US2019/060131, titled “Systems and Methods for Skin Treatment”, filed Nov. 6, 2019 [Docket Number 2012312-0261, CYT-010-PCT].

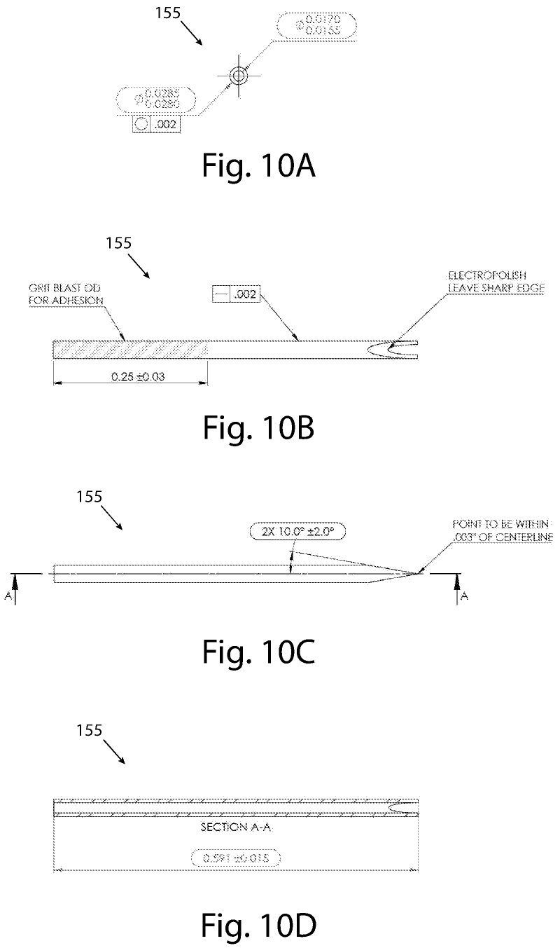

Coring elements 155 can comprise one, two or more hollow filaments, such as coring element 155 described herein in reference to FIGS. 10A-D. Each coring element 155 can comprise an elongate shaft (e.g. a hollow shaft), shaft 1551 shown, which can include a distal end. Each coring element 155 can comprise one or more projections, prong 1552 shown, that extend from the distal end of shaft 1551.

Spacer assembly 180 can comprise a housing and other components that are configured to properly position treatment module 150 relative to the patient's skin being treated. Spacer assembly 180 can include one or more sensors, sensor 181 shown, which can be configured to detect proper engagement of spacer assembly 180 with the patient (e.g. proper pressure level detected).

Actuation assembly 120 can be configured to interface with treatment module 150 by performing a function selected from the group consisting of: control the motion of a treatment module 150 (e.g. translate treatment module 150 along one, two, or three axes); activate one or more components of treatment module 150 (e.g. advance and/or retract one or more coring elements 155 into and/or from tissue); rotate one or more components of treatment module 150 (e.g. rotate one or more coring elements 155 prior to, during, and/or after their insertion into tissue); vibrate one or more components of treatment module 150; and combinations of these. Actuation assembly 120 comprises actuator 121 shown. Actuator 121 and other components of actuation assembly 120 can be of similar construction and arrangement as the similar components described in applicant's co-pending PCT application Serial Number PCT/US2019/060131, titled “Systems and Methods for Skin Treatment”, filed Nov. 6, 2019 [Docket Number 2012312-0261, CYT-010-PCT]

Console 500 can comprise user interface 510 as shown, which can comprise one or more user input and/or user output components, such as one, two or more components selected from the group consisting of: display; touch screen display; button; switch; foot switch; lever; membrane keypad; mouse, joystick; microphone; speaker; vibrational and/or other haptic transducer; light such as a light emitting diode; and combinations of these. Console 500 can comprise controller 520 as shown, which can include: one or more central processing units (CPUs), microprocessors and/or other microcontrollers, processor 521 shown; memory 522 shown (e.g. volatile or non-volatile memory); instructions 523 shown; signal processing and other electronic circuitry; oscillator circuitry such as voltage-controlled oscillator (VCO) circuitry; analog to digital circuitry; digital to analog circuitry; and/or other componentry configured to control or otherwise interface with one or more components of system 10. Controller 520 can comprise a power supply and/or energy storage component (e.g. a battery, a capacitor, and/or a power supply converted to receive “wall power” and convert it to an AC or DC voltage for use by system 10). Console 500 can further comprise drive module 550, and vacuum assembly 560, each as shown. Console 500 and its various components can be of similar construction and arrangement to those described in applicant's co-pending PCT application Serial Number PCT/US2019/060131, titled “Systems and Methods for Skin Treatment”, filed Nov. 6, 2019 [Docket Number 2012312-0261, CYT-010-PCT].

As used herein, a “treatment plan” comprises a set of parameters that are used in treating target tissue of the patient using system 10. A treatment plan can include a set of treatment settings, such as one, two or more microcoring parameters. A treatment plan can include a set of different medical procedures (e.g. one, two or more microcoring procedures and/or other treatment procedures). A treatment plan can include a desired and/or recommended order for performing a set of multiple medical procedures (e.g. where the treatment plan provides multiple procedures to be performed in a particular order, where in some instances sufficient efficacy is achieved when a subset of the procedures is performed). In some embodiments, system 10 is configured to automatically and/or semi-automatically (“automatically” herein) generate a treatment plan (e.g. one or more treatment plans made available to a clinician). System 10 can generate a treatment plan using an algorithm, such as algorithm 525 described herein. A treatment plan can be developed by algorithm 525 using at least image data ID, such as by using image data ID comprising: ultrasound-based image data (e.g. Doppler data and/or other image data produced using ultrasound); CT-based image data; MRI-based image data; and/or X-ray-based image data (e.g. fluoroscopic data and/or other image data produced using X-ray). Alternatively or additionally, algorithm 525 can develop a proposed treatment plan based on parameters selected from the group consisting of: patient age; patient race; patient gender; patient skin type; patient skin condition; volume of target tissue to be treated; cellulite and/or fat content of target tissue; geometry of target tissue; tissue type, geometry and/or other characteristic of non-target tissue proximate the target tissue; and combinations of these. In some embodiments, a treatment plan includes a methodology to ensure treatment of target tissue, while avoiding damage to neighboring non-target tissue. In some embodiments, system 10 (e.g. via algorithm 525) is configured to produce a prediction of outcome (e.g. an estimation of likelihood of efficacy and/or an assessment of any risks) associated with one or more treatment plans.

System 10 can comprise algorithm 525 shown, which can comprise one or more algorithms. All or a portion of algorithm 525 can be integrated into one, two or more of various components of system 10, such as console 500 (as shown), treatment device 100, imaging device 50, TCA 600, and/or functional element 99. Algorithm 525 can comprise one or more machine learning, neural network, and/or other artificial intelligence algorithms (“AI algorithm” herein).

Algorithm 525 (e.g. an AI algorithm) can be configured to determine and/or modify one or more microcoring parameters, such as to effectively treat target tissue (e.g. improve cosmesis of the patient) and/or avoid damage to non-target tissue. For example, algorithm 525 can be configured to determine a volume of target tissue to be treated (e.g. treated with a microcoring procedure), such as to effectively enhance cosmesis of the patient and/or otherwise provide a therapeutic benefit to the patient, while avoiding or at least minimizing damage to non-target tissue. In these embodiments, algorithm 525 can be further configured to determine and/or modify one or more microcoring parameters (e.g. at least based on the determined volume), such as to effectively treat the target tissue volume determined, while avoiding damage to non-target tissue, as described hereabove.

Algorithm 525 can be configured to perform a “microcoring analysis” comprising using an analysis of one or more types of information by algorithm 525 to assess the level of microcoring (e.g. the current level of microcoring) of target tissue. The results of this analysis can be used by system 10 to perform microcoring in a closed loop mode. Microcoring data produced in the microcoring analysis can be stored as image data ID (e.g. and correlated with one or more tissue locations). In some embodiments, system 10 (e.g. treatment device 100 and/or imaging device 50) delivers and/or receives energy (e.g. light energy and/or ultrasound energy or other imaging-capable energy) to and/or from tissue, and algorithm 525 performs a microcoring analysis based on the delivered and/or received energy.

Algorithm 525 can be configured to adjust tissue treatment parameters (e.g. microcoring parameters) based on sensor signals, such as when sensor 199a provides feedback to algorithm 525 regarding a microcoring procedure.

In some embodiments, algorithm 525 is configured to perform an analysis on patient data PD (e.g. patient use data from a single patient, or a group of patients upon which system 10 has performed a treatment procedure), such as to modify a future treatment provided by system 10.

In some embodiments, algorithm 525 is configured to provide a treatment plan, such as when algorithm 525 performs analysis on patient data PD comprising data collected during treatment of the patient with system 10 in a previous treatment procedure, and/or based on patient data PD collected from use of system 10 on multiple patients (e.g. a large number of patients treated with system 10).

Algorithm 525 can comprise one or more algorithms that are performed by processor 521 of controller 520. Processor 521 can perform algorithm 525 using instructions 523, such as instructions 523 that are stored in memory 522 of controller 520.

System 10 can include network 80 as shown, which can comprise one or more computer networks such as the Internet, a local area network, cellular network, and/or other data sharing, storage, and/or transmitting platform. In some embodiments, patient data PD, and/or other data collected during the use of system 10 is transmitted from one location to another location over network 80. In some embodiments, one or more central data storage areas are used to store the data, such as when an algorithm 525 analyzes the data to provide a treatment plan and/or provide system 10 parameters for a future treatment of one or more patients.

Treatment device 100 and/or another component of system 10 can be configured to perform a treatment (e.g. a microcoring treatment) in a closed loop mode (i.e. a closed loop mode of microcoring and/or other closed loop mode of operation), such as when one or more sensors of system 10 (e.g. a sensor-based functional element 99, 199, and/or 599), provide patient and/or system information that is used to continuously and/or intermittently adjust the treatment being delivered by treatment device 100 (e.g. adjust the microcoring parameters and/or other parameters of the treatment). For example, microcoring can be adjusted in a closed loop mode based on a system 10 parameter and/or based on a patient parameter (e.g. a patient physiologic parameter, patient anatomical parameter, and/or a patient environment parameter, each as described herein). Microcoring by treatment device 100 can be adjusted based on image data ID described herein, such as to redirect and/or otherwise adjust microcoring (e.g. due to detected patient motion and/or undesired treatment device 100 motion) and/or to change one or more microcoring parameters (e.g. as determined by algorithm 525 using image data ID or other data). In some embodiments, image data ID is used to determine when a treatment (e.g. a microcoring amount) is sufficient, such as when algorithm 525 analyzes image data ID to confirm sufficient change in tissue characteristics have occurred.

As described herein, system 10 can be configured to perform a series of clinical procedures on a patient, such as a patient desiring improved cosmesis of the face or other body location, as described herein. In some embodiments, system 10 is configured to be used to: perform a first procedure and a second procedure, in which the two procedures are performed at least 24 hours apart. The first procedure can include microcoring, the second procedure can include microcoring, or both can include microcoring. In some embodiments, the first procedure does not include microcoring, while the second procedure does include microcoring. In some embodiments, two, three, four, or more microcoring procedures of the present inventive concepts are performed, such as over a period of months and/or years. In some embodiments, the treatment plan for a subsequent procedure using system 10 is based on the data collected and/or results of one or more previous treatment procedures performed using system 10.

System 10 can be configured to perform a treatment on a patient (e.g. a patient desiring improved cosmesis of the face or other body location) that includes the performance of multiple, sequential treatment plans, such as a sequence of treatment plans that each may use one, two or more components of system 10 (e.g. one, two or more of treatment devices 100) that are used to perform one or more diagnostic procedures, and/or one or more therapeutic procedures. Performance of an “initial treatment plan” performed using system 10, can be configured based on current physiologic state (e.g. current undesired state of tissue) of the patient, as well as any previous treatments performed (e.g. using system 10 or otherwise). Each “subsequent treatment plan”, can also be based on the current physiologic state, as well as all previous treatments performed, as described herein.

In some embodiments, the one or more coring elements 155 (e.g. three coring elements 155) comprise a dimension selected from the group consisting of: an outer diameter of no more than 0.050 in, or no more than 0.040 in, such as approximately 0.028 in; an inner diameter of no more than 0.030 in, or no more than 0.025 in, such as approximately 0.016 in; a core length of at least 0.5 mm and/or no more than 5.0 mm; a penetration depth of no more than 6.0 mm; a cutting depth of no more than 5.0 mm; and combinations of these.

In some embodiments, one or more coring elements 155 comprise a double-beveled needle geometry (e.g. as shown in FIGS. 10A-D), such as to minimize effective insertion depth and/or resist wear during use.

In some embodiments, system 10 is configured to precisely control insertion speed of the one or more coring elements 155 (e.g. simultaneous insertion of all of coring elements 155). In these embodiments, the dwell time can comprise a time of no more than 60 msec, such as no more than 45 msec, no more than 30 msec, and/or no more than 20 msec. System 10 (e.g. console 500 and/or treatment device 100) can comprise a proportional integral derivative (PID) controller that provides closed loop control of coring element 155 advancement and position that results in accurate core depth, such as while minimizing impact forces on the patient's skin (e.g. thus improving healing response and core hole precision).

In some embodiments, multiple coring elements 155 are positioned in an array (e.g. a linear arrangement of three or four elements 155) in which the coring elements 155 are separated by a distance of at least 0.2 mm, such as at least 0.5 mm, at least 1.0 mm, at least 2.0 mm, and/or approximately 3.33 mm.

System 10 can include tissue collection assembly 600 for clearing tissue cores captured by coring elements 155. In some embodiments, LPS 650 comprises a single source of low pressure (e.g. vacuum) that provides multiple (e.g. two) functions. System 10 can be configured to control the flow rate (e.g. the pressure) proximate the coring elements 155, such as to remove tissue cores without impacting low pressure applied to spacer assembly 180 (e.g. spacer assembly 180 using suction to stabilize treatment module 150 relative to the patient's skin). The flow channels into which the tissue cores are extracted can include a funnel portion that increases the flow velocity at locations where the tissue is extracted from the back ends of the coring elements 155.

Treatment device 100 can comprise spacer assembly 180, which can provide a stabilizing force to treatment device 100 during use, as described herein. For example, spacer assembly 180 can utilize a suction force that allows effective treatment of target tissue areas comprising various surface contours. System 10 can include an automated pinch valve in line with vacuum conduits provided to spacer assembly 180, such as to provide enhanced stabilization of treatment module 150 with the patient's skin between patterns of deployment of one or more coring elements 155. For example, the pinch valve can be activated to allow easy repositioning of treatment module 150 (e.g. and spacer assembly 180) at the end of a pattern of microcoring, such as to improve ease and speed of a treatment.

Treatment device 100 can comprise a “treatment window” that is sized to accommodate various ranges of suction force to be applied. In some embodiments, spacer assembly 180 provides a treatment window of at least 100 mm², such as no more than 2,000 mm², such as approximately 640 mm², such as to provide a nominal holding force of treatment module 150 (e.g. spacer assembly 180) of at least 10.0N, such as at least 18.0N, such as approximately 28.5N with the patient's skin.

System 10 can be configured to detect (e.g. and quantify) deceleration of coring elements 155, such as to minimize damage to the coring elements 155 and/or to detect damage to at least one coring element 155.

System 10 can include various features that enhance positioning accuracy (e.g. during deployment) of coring elements 155, such as positioning accuracy in X and Y directions, and/or positioning accuracy in the Z dimension (e.g. insertion direction). Such features include but are not limited to: 1:1 gearing and/or direct drive-in actuation assembly 120; sensor detection of position (e.g. hall sensors and/or optical sensors such as optical encoders); linear bearings (e.g. that minimize undesired motion and/or creep from a desired position); and combinations of these.

System 10 can be configured to provide variable patterns for microcoring (e.g. varied microcoring density), such as to achieve a skin removal percentage (also referred to as “areal fraction”) of no more than 20%, and/or no less than 0.5%, such as at least 1%, and/or at most 10% (e.g. between 1% and 10%).

Actuation assembly 120 can comprise one or more actuators (e.g. solenoids) that are configured to precisely control movement of one or more coring elements 155 such as to achieve variable depth control within 0.8 mm, such as within 0.5 mm, while accommodating variability in skin thickness, skin toughness, and/or other varying skin parameters.

System 10 can comprise a calibration routine such as to store calibration information created during manufacturing of one or more components of system 10, and/or information collected at a clinical site (e.g. prior to, during, and/or after use of system 10). Calibration data can be stored in a treatment module 150, actuation assembly 120, and/or other component of treatment device 100. System 10 can be configured to improve accuracy of needle deployment (e.g. in the Z direction), based on the calibration data (e.g. to accommodate variability in manufacturing processes).

Coring elements 155 can comprise a bevel angle of no more than 30 degrees, such as no more than 25 degrees, and/or no more than 20 degrees, such as to improve healing and/or minimize scarring of the patient.

System 10 can be configured to control the speed and/or frequency (e.g. repetition rate) of the deployment of the coring elements 155 into the patient's skin, such as to deploy the elements 155 (e.g. three elements 155 in unison) at a rate of at least 1 Hz, or 3 Hz, or approximately 8 Hz. Alternatively or additionally, system 10 can be configured to deploy the elements 155 (e.g. three elements 155 in unison) at a rate of no more than 30 Hz, such as no more than 20 Hz, such as approximately 8 Hz.

System 10 can be configured to perform various aesthetic procedures on patients, such as a microcoring procedure (as described herein) in which excess skin (e.g. associated with aging) is removed, without invasive surgery and without evidence of scarring. System 10 can be configured to perform one or more aesthetic procedures without use of thermal energy (e.g. without causing any significant increase in tissue temperature), such as to provide an accelerated healing response (e.g. as compared to energy-based systems). System 10 can be configured to remove microcores of dermal and/or epidermal tissue for the treatment of moderate to severe wrinkles (e.g. in the mid and lower face without surgery). In some embodiments, algorithm 525 is configured to adjust one or more system 10 operational parameters (e.g. microcoring parameters) in order to achieve a desired therapeutic outcome, such as one, two, or more therapeutic outcomes as described herein. The adjustment of the parameters by algorithm 525 can be performed automatically. The adjustment of the parameters by algorithm 525 can be performed based on one or more patient parameters recorded by one, two or more sensors of system 10 and/or one or more patient parameters provided to system 10 via user interface 510.

In some embodiments, a microcoring procedure performed using system 10 (e.g. as described herein) is configured to achieve a therapeutic outcome comprising a physiologic effect selected from the group consisting of: adipose tissue remodeling and/or removal; dermal remodeling; dermal tightening; and combinations of one or more of these.

In some embodiments, a microcoring procedure performed using system 10 is configured to achieve a therapeutic outcome comprising a physiologic improvement selected from the group consisting of: maintenance and/or remodeling of elastin; procollagen and/or collagen production; skin appearance, such as skin appearance that has been decreased by menopause; skin barrier repair and/or function; skin contour appearance; skin elasticity; skin luminosity; skin moisture; skin plumpness; skin softness; skin suppleness; skin tautness; skin texture and/or promotion of re-texturation; skin thickness; skin tone, radiance, and/or clarity; skin elasticity and/or resiliency; and combinations of one or more of these.

In some embodiments, a microcoring procedure performed using system 10 is configured to achieve a therapeutic outcome comprising the inhibition in the appearance of wrinkles.

In some embodiments, a microcoring procedure performed using system 10 is configured to achieve a therapeutic outcome comprising the modification of a hair follicle, such as to prevent the growth of hair and/or inhibit the growth of hair.

In some embodiments, a microcoring procedure performed using system 10 is configured to achieve a therapeutic outcome comprising reducing a physiologic feature selected from the group consisting of: an acne scar; a cheek wrinkle; a dynamic wrinkle, fine wrinkle, and/or static wrinkle; an eye wrinkle; elastosis; a facial pore; a pigment spot; sebaceous gland activity; size of a wrinkle; a stretch mark; a surgical scar; a tattoo; and combinations of one or more of these.

In some embodiments, a microcoring procedure performed using system 10 is configured to achieve a therapeutic outcome comprising reducing a tattoo (e.g. reducing the visual appearance of a tattoo).

In some embodiments, a microcoring procedure performed using system 10 is configured to achieve a therapeutic outcome comprising regenerating skin (e.g. facial skin).

In some embodiments, a microcoring procedure performed using system 10 is configured to achieve a therapeutic outcome comprising the replenishing of (e.g. causing the replenishment of) essential nutrients, and/or constituents in the skin.

In some embodiments, a microcoring procedure performed using system 10 is configured to achieve a therapeutic outcome comprising restoring skin luster and/or skin brightness.

In some embodiments, a microcoring procedure performed using system 10 is configured to achieve a therapeutic outcome comprising treating and/or reducing a physiologic feature selected from the group consisting of: fine lines and/or wrinkles; one or more scars; skin sagging; and combinations of one or more of these.

Applicant has conducted various studies using the systems, devices, methods, and other technologies of the present inventive concepts, such as system 10 and its components as described herein. System 10 can be configured to remove skin via microcoring, such as without use of thermal energy (e.g. avoiding damage to cells from heating) during the microcoring procedure. Energy-based devices such as fractional laser and radiofrequency ablation lead to epidermal and dermal cell necrosis from thermal injury that may inhibit rapid wound closure, an adverse effect that can be avoided via use of system 10. Although fractional lasers and radiofrequency devices have shown acceptable results in rejuvenation of skin, data on skin tightening is inconclusive. It is suspected that coagulation necrosis of the cells surrounding fractional laser cores prevent early wound closure and therefore limit reduction of skin surface area and skin tightening. System 10 avoids coagulation necrosis and can achieve both early wound closure, and enhanced skin tightening, as described herein. The coring elements 155 and other components of system 10 provide numerous benefits including limited side effects, and fast (e.g. expedited) patient recovery. By removing tissue, significant skin tightening can be achieved, as demonstrated by data gained in clinical procedures performed on human patients.

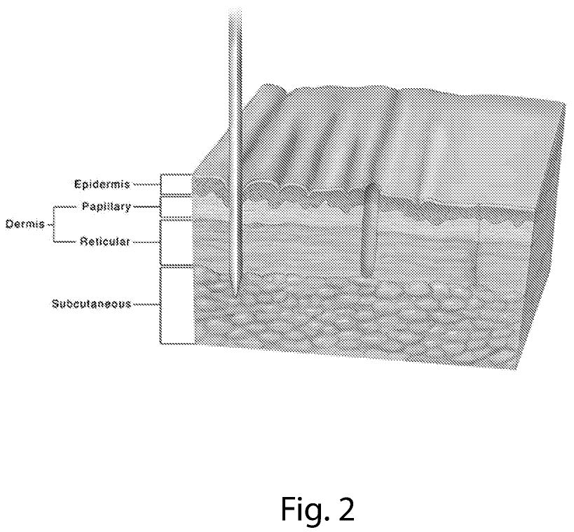

System 10 can be configured to both tighten skin and reduce skin wrinkles and/or folds of the patient's skin. Use of system 10 in human patients has achieved skin tightening as well as reduction in skin wrinkles and/or folds, via removal of skin without the use of thermal energy, while also reducing (e.g. preventing or resulting in minimal) scar formation. FIG. 2 illustrates a coring element 155 being safely introduced into the skin, such as to subsequently be withdrawn to remove a microcore of tissue, such that the remaining tissue heals with no scarring or at most minimal scarring. The treatment provided by system 10 also provides near-immediate closure along the relaxed skin tension lines (RSTLs), with no thermal energy.

Applicant has also conducted non-human mammalian studies that resulted in an improved safety and efficacy profile (e.g. as compared to thermal treatment and other commercially available treatments). Studies performed using system 10 in porcine skin demonstrated that wound healing in the treatment areas was achieved after one week and erythema was completely resolved at two weeks, with no evidence of infection or scarring over a three-month period. At one month, a significant increase in epidermal and papillary dermal thickness was seen. Further, collagen content increased by 89% at three months. System 10 can be configured to cause a collagen content increase of at least 30%, 50%, and/or 70% (e.g. an increase present at a time frame of 3 months after performance of the microcoring procedure performed using system 10). Both skin thickening and increased collagen content indicate histologic endpoints of successful skin rejuvenation. In addition, porcine skin treated by treatment device 100 at a 10% microcoring density with a coring element 155 comprising a 19 Gauge coring needle exhibited a reduction in skin surface area by 9.4%+4.3 as compared to 3% in control areas treated with standard hypodermic needles with no tissue removal (p<0.01). This finding confirms skin tightening after microcoring procedures performed using system 10.

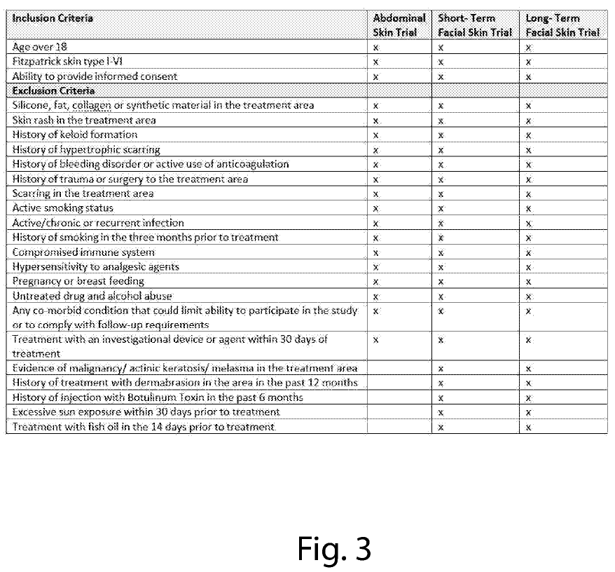

Based on the encouraging results of these porcine studies and other research, applicant conducted three clinical trials to evaluate the safety using system 10 to perform a microcoring treatment of the present inventive concepts in human skin. The primary goal of the three studies was to determine safe system 10 treatment parameters, evaluate the healing profile of human skin after microcoring using system 10, and analyze safety of system 10. In addition, efficacy variables were assessed. The trials included various inclusion and exclusion criteria as shown in Table 1 of FIG. 3. The findings of all three trials are summarized herebelow.

### Clinical Trial Methods

All three of these human clinical safety trials were approved by the New England Institutional Review Board as non-significant risk medical device studies. All patients signed informed consent adhering to the guidelines outlined by the International Conference on Harmonization (ICH) Good Clinical Practice (GCP). No patients were lost to follow-up. No antiviral prophylaxis was prescribed, and patients were not given prophylactic antibiotic or antiviral therapy after treatment.

Safety parameters were evaluated for all three clinical trials at all timepoints and consisted of: patient reported pain (on a scale of 0-10), bleeding (classified as: none, trace, mild, moderate, severe), healing profile (classified as: presence of ecchymosis, purpura, fluid accumulation, hyperpigmentation, hypopigmentation, roughness, dryness, inflammation, erythema, crusting on a scale of 0-absent, 1-trace, 2-mild, 3-moderate to 4-severe), scarring (classified as yes/no), and adverse events.

### Abdominal Skin Trial

In order to evaluate system 10 for reduced scar (e.g. scar-less) skin removal in human skin, applicant designed a prospective, randomized controlled in-human feasibility trial. Five patients scheduled to undergo abdominoplasty surgery 90 days after enrollment in the study were treated with system 10 in the area to be removed during the abdominoplasty operation. 1 cm by 1 cm treatment areas and untreated control areas were marked by permanent tattoo prior to the microcoring procedure performed using system 10. Patients were randomized to system 10 size of coring elements 155 needle gauge, with diameters ranging from 19G to 24G, and microcoring density between 10% and 20% of the skin surface area to be treated (i.e. the marked skin surface area).

As an additional safety variable, two patients underwent tissue biopsy on day 90 to confirm absence of scarring on histology. All safety endpoints were evaluated per target tissue treatment area on day 0 and days 1, 7, 30, 60, and 90 post treatment and compared to the untreated control areas.

To determine a possible skin rejuvenation effect, skin thickness was evaluated with a skin analysis system (DermaLab Combo® system) on day 90 and compared to the untreated control areas. Descriptive statistics, and T tests were used for analysis.

### Short-Term Facial Skin Trial

To determine whether use of system 10 is appropriate and safe for use in the face of a patient, a 30 day prospective randomized controlled single-blind clinical trial for use of system 10 in the preauricular area was designed and conducted. Nine patients were randomized to receiving microcoring treatment using system 10 in a 2 cm by 1 cm area of skin in the preauricular area 30 days prior to excision during facelift surgery. One patient was screened but did not undergo treatment. The system 10 target tissue treatment area and untreated control areas were marked by permanent tattoo prior to the intervention. Microcoring density was fixed at 10% of the treatment area, whereas size of coring elements 155 was randomized to either 22G or 24G.

Based on findings in the abdominal skin trial, erythema and melanin content were evaluated with optical reflectometry as an additional safety variable. All safety endpoints were evaluated per treatment area on day 0 after treatment, as well as days 1, 7, 15, and 30, and compared to the untreated control areas. Efficacy outcomes included change in skin thickness (e.g. as assessed using DermaLab Combo® skin analysis system), and reduction of skin surface area (measured by analysis of the skin surface area between tattooed points via stereo photogrammetry on three-dimensional images obtained with the Canfield Vectra H1 handheld camera). Efficacy endpoints were analyzed at 30 days. Descriptive statistics, and paired T tests were performed for analysis.

### Long-Term Facial Skin Trial

In order to determine whether a system 10 microcoring treatment is safe over an extended period of time, a 90-day prospective single-blind, randomized bilateral paired comparison study in human preauricular facial skin was designed and conducted. A total of 15 patients (30 treatment sites) were randomized to system 10 microcoring treatment in a 2 cm by 1 cm area of skin in the preauricular area that was not removed surgically. Treatment parameters were randomized to each treatment area with the size of coring elements 155 ranging between 22, 24 and 25G, and microcoring density of either 2.5%, 5%, 7.5%, or 10%.

As an additional safety variable, scarring was evaluated using the Manchester Scar Scale-MSS. Safety outcome variables were evaluated at every timepoint. All efficacy endpoints were evaluated at 30 and 90 days. Efficacy outcome variables included overall aesthetic improvement which was measured by patients and investigators and reported using the global aesthetic improvement scale (GAIS; where 3=very much improved; 2=much improved; 1=improved; 0=no change; −1=worse; −2=much worse; −3=very much worse). Study visits were conducted on days 0, 1, 4, 7, 15, 30, 90. Descriptive statistics, and paired T tests, were performed for analysis. System 10 can be configured to perform a microcoring procedure, as described herein, and achieve minimal scarring, such as scarring that results in a Manchester Scar Scale value of less than 10 (i.e. no scarring to mild scarring).

### Results

### Demographics

All patients were Caucasian. In the abdominal skin trial, five female patients with Fitzpatrick skin type 1-3 and a mean age of 46 (+11 years with a minimum age of 34, and maximum age of 58) were included. The short-term facial skin trial included seven female and two male patients with Fitzpatrick skin type 1-3. Average age was 64.5 (±3.6) with a minimum age of 58 and a maximum age of 71. Eight female and seven male patients with Fitzpatrick skin type 1-5 were enrolled in the long-term facial skin trial. Mean patient age was 56.2±6 years with a minimum age of 44 and maximum age of 64.

### Technical Device Safety

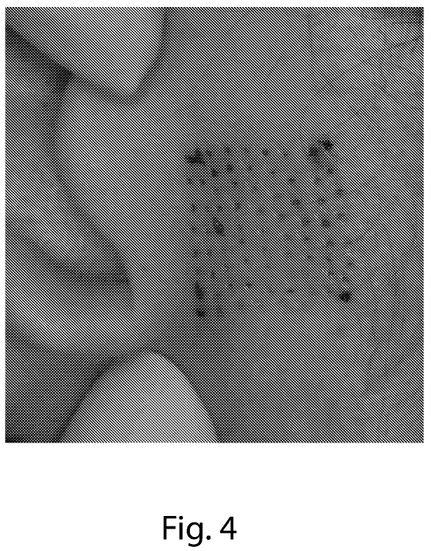

For all studies, system 10 operated as clinically intended and patterns of micro-excisions were generated in abdominal skin, as well as facial skin with microcoring elements (e.g. coring elements 155 herein) comprising needles of 19G to 24G (abdomen) and 22G to 25G (face). No system 10 safety events were reported in any of the three clinical trials. Reference FIG. 4 for an example of a system 10 treatment area immediately after microcore removal. Microcores of skin have been removed resulting in microscopic circular wounds.

### Clinical Safety Profile

### Adverse Events and Serious Adverse Events

In the short-term facial skin trial, one patient developed a superficial wound infection in the preauricular area 21 days after treatment that resolved without intervention. No other adverse events or serious adverse events were reported in any of the three clinical trials.

### Pain

Across all treatment areas on abdominal skin, the average pain during treatment was 2.8±1.1 on a scale of 0-10. Pain decreased to 0.4±0.9 on day 1 and 7, and to 0 on all subsequent visits. Average pain in the short-term facial skin trial was 0.4±1 during treatment and 0.4±1.3 on day 1, 1±2 on day 7 and 0.2±0.7 on day 30. In the long-term facial skin trial, pain during treatment was reduced to 0±0. Mean post-procedure pain was 0.6±0.92 on day 1, 0.4±0.8 on day 3, 0.07±0.37 on day 7, 0.2±0.6 on day 15, and 0±0 starting day 30 and on all subsequent visits. System 10 can be configured to perform a microcoring procedure on a patient's abdomen, and the patient can experience (e.g. during the procedure) a pain level, as measured on a scale of 0-10, that is less than 5, less than 4, and/or less than 3. Alternatively or additionally, system 10 can be configured to perform a microcoring procedure on a patient's face, and the patient can experience (e.g. during the procedure) a pain level, as measured on a scale of 0-10, that is less than 4, less than 3, less than 2, and/or less than 1.

### Bleeding

Bleeding during treatment of the abdominal skin was trace in two patients and mild in three patients. During the short-term facial skin trial, seven patients experienced mild bleeding during treatment and two patients had moderate bleeding. Analysis of the long-term facial skin trial data revealed no bleeding at three (10%) treatment sites, trace at 23 (77%) treatment sites, and moderate at four (13%) treatment sites.

### Healing Profile

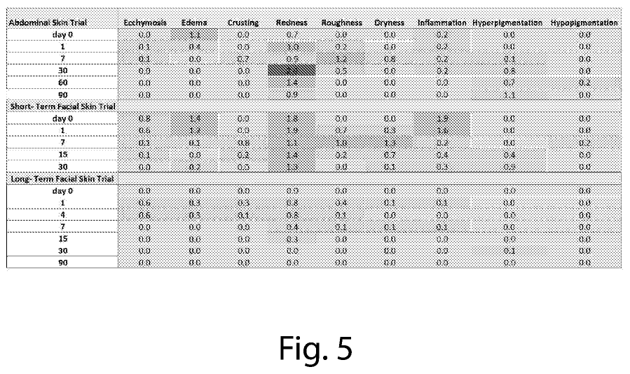

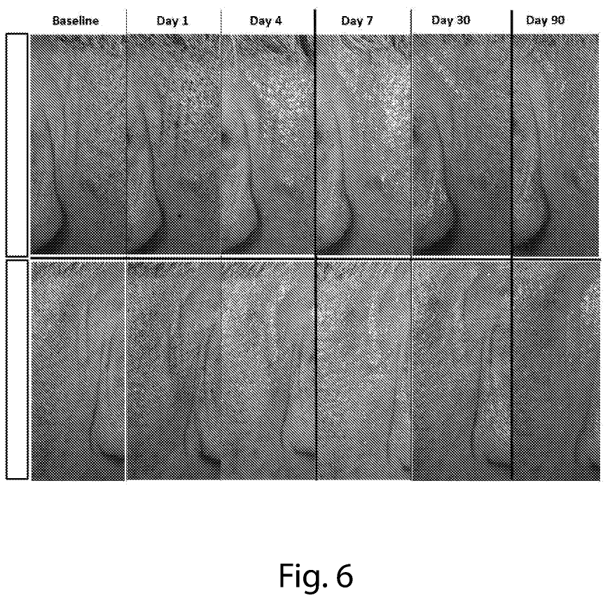

Referring to FIGS. 5 and 6, system 10 treated skin healed with no observable scarring. FIG. 5 provides Table 2, a table of average score healing profiles (classified as: 0—absent, 1—trace, 2—mild, 3—moderate, and 4—severe) across all patients at various days after treatment by system 10. FIG. 6 illustrates photographs of treatment sites of patients that exhibit wound healing after system 10 treatment. Most treatment side effects resolved by day 7. Also of note was the clinical improvement achieved in rhytides at day 90.

During the abdominal skin trial, trace to mild treatment side effects such as ecchymosis, edema, crusting, roughness, dryness, and inflammation were seen up to day 30. Trace to mild redness was present from day 1 to day 90. Trace hyperpigmentation was seen on days 7-90.

The short-term facial skin clinical trial showed trace ecchymosis, crusting, and roughness up to day 15. Trace edema, redness, dryness, inflammation, and hyperpigmentation were present up to day 30.

During the long-term facial skin trial trace side effects (roughness, dryness, inflammation) were noted up to day 7. Trace redness was seen on days 1-15 and was absent on day 30 and subsequent visits. Trace hyperpigmentation was seen on day 30 that resolved by day 90.

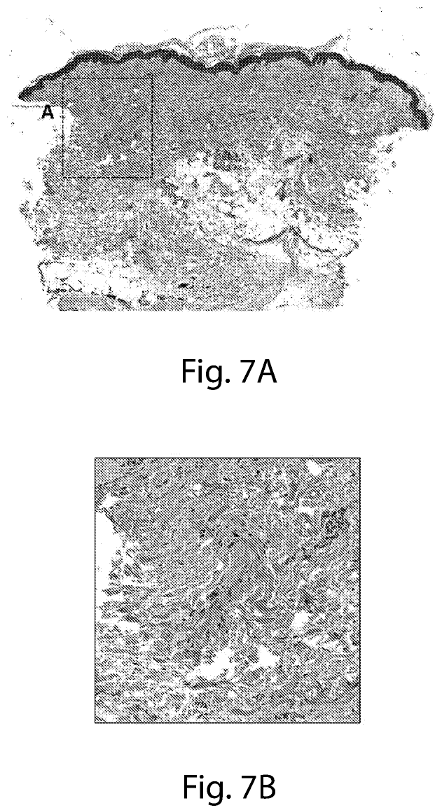

Additional healing parameters included histologic evaluation of biopsies obtained in two patients during the abdominal skin trial at the day 90 visit that confirmed absence of scarring at 10% microcoring density, as shown in photographs of histological slides shown in FIGS. 7A-B. Further, the erythema index that was calculated as part of the short-term facial skin trial showed no significant difference between treatment (22.7±7.1) and control (25±6.2) groups on day 30 (p=0.16). In addition, there was no significant difference in melanin index between groups (treatment 40.3±5.7 versus control 43.6±5.3; p=0.12).

In the long-term facial skin trial, Manchester Scar Scale evaluation revealed no scarring in any treatment area (n=30) with microcoring elements (e.g. coring elements 155 herein) with a size of 22G to 25G and a 2.5% to 10% microcoring density in the preauricular area.

### Clinical Efficacy Profile Both Abdomen and Face Skin Thickness Increase

### Skin Thickness

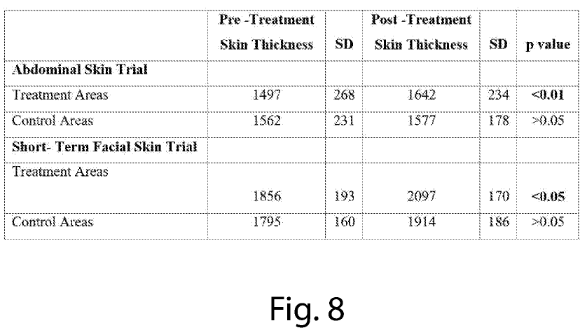

Both abdominal and facial skin thickness increased after microcoring treatment performed using system 10, as shown in Table 3 of FIG. 8. Analysis of the abdominal skin treatment sites revealed a significant increase in skin thickness in treated areas of the abdomen as compared to control areas from baseline to 90 days post treatment (p<0.01). An increase in skin thickness could also be seen for treated facial skin as compared to control (p<0.05).

### Skin Surface Area Reduction

In the short-term facial skin trial, skin surface area reduction at 10% microcoring density with coring element 155 size at 22G and 24G was on average −9.4%±4.3% (−13.9±7 mm2), which was significant as compared to baseline and control (p<0.01). System 10 can be configured to perform a microcoring procedure and achieve a skin surface area reduction of at least 3%, 5%, and/or 7% of the area treated.

### Aesthetic Improvement

Analysis of the long-term facial skin trial data revealed that the mean patient GAIS score was 2.9±0.6 and mean investigator GAIS was 2.8±0.5 at 90 days, a significant improvement. There was a visible reduction of rhytids on clinical exam (e.g. as shown in FIG. 6).

### DISCUSSION

A system 10 using coring elements 155 comprising hollow filaments is configured to remove skin resulted in no observable formation of scar tissue. Studies demonstrated that use of system 10 to perform microcoring of abdominal and facial skin is well tolerated with minimal pain and bleeding during treatment. Use of system 10 is safe and provides an excellent healing profile, and its use shows signs of skin rejuvenation such as increase in abdominal and facial skin thickness, skin surface area reduction (skin tightening), and global aesthetic improvement.

Microcoring treatment provided by system 10 was well tolerated with only mild pain during and after the procedure. Minimal pain was reported during treatment of the abdomen (2.8±1.1) and facial skin (0±0-0.4±1.3). The pain profile improved as coring specifications, parameters, and/or techniques were refined from the abdominal skin trial to the facial skin trials. Improved coring specifications, parameters, and/or techniques included one or more of: needle sharpness; replacement of needles such as due to wear; use of vacuum to stabilize the skin before punctures; transitioning from hand puncturing of needles to power-driven advancement of needles; and combinations of these. A recent review demonstrated that patients undergoing current micro-needling procedures experienced pain levels on average 0.2 to 3.8 out of 10, which is slightly higher than that experienced using system 10 in the human clinical studies reported herein. Therefore, pain levels during microcoring treatment using system 10 are comparable or lower than pain reported with micro-needling.

Further, transient and self-limited bleeding was observed during treatment that was well tolerated by patients and did not require additional hemostasis. Similar to micro-needling, pinpoint bleeding is the endpoint of system 10 treatments, and the amount of bleeding that was seen in all three clinical trials (trace to moderate) was within the expected, acceptable range.

System 10 treated skin demonstrated a favorable healing profile with no signs of clinical or histologic scarring. This finding confirms that scar-less skin removal with system 10 can be achieved in human skin. Expected treatment side effects were observed with trace to mild severity across all clinical trials. The side effect profile improved with refined coring parameters and treatment technique, such as is described hereabove. During the final long-term facial skin trial with improved coring specifications and technique, trace side effects such as ecchymosis and edema were present up to day 7 with only trace redness persistent until day 15 and one instance of trace hyperpigmentation on day 30 that resolved by day 90. With fractional carbon dioxide laser resurfacing, a similar short-term healing profile can be observed with most patients experiencing side effects for approximately 14 days. However, long-term side effects such as hyperpigmentation and hypopigmentation are less common with use of system 10.

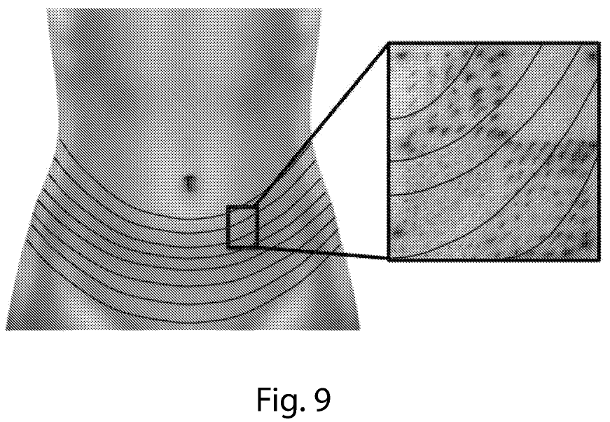

Healing after treatment with system 10 occurred predominantly along the prevailing RSTLs. After system 10 treatment of abdominal skin, the tattooed round cores appeared elliptical along the RSTLs at 90 days post treatment, as shown in FIG. 9. RSTLs are furrows that are created when the skin is relaxed in absence of tension. Therefore, surgical incisions are ideally oriented along the RSTLs, as it is well known that wounds heal most inconspicuously under no tension. The observation that microcores created by system 10 heal along the RSTLs is very encouraging, as this means ideal and aesthetic wound healing occurs.

Use of system 10 in these clinical studies exhibited notable signs of skin rejuvenation. Preliminary findings include a significant increase in skin thickness in system 10 treated areas as compared to control in both abdominal and facial skin. One of the main characteristics of aging skin is decreased collagen production that leads to thinning of the epidermis and dermis. Increase in skin thickness suggests an increase in collagen production and reversal of aging effects.

The average reduction of the facial skin surface area using system 10 was −9.4%+4.3 at 10% microcoring density on post procedure day 30, which was significant as compared to baseline and control (p<0.01). During treatment with fractional devices that use thermal energy, coagulation necrosis of the cells surrounding fractional laser cores occurs seemingly inhibiting closure of micro wounds. With system 10, closure along the RSTLs occurred within 24 hours of treatment, without visible interposition of debris. The absence of coagulation necrosis using system 10 can allow for effective reduction in skin surface area and skin tightening.

Both patients and blinded investigators felt that the overall aesthetic improvement of the system 10 microcoring treatment areas in the long-term facial skin trial was very much improved, indicating a positive effect overall.

In summary, microcoring treatment using system 10 has been shown to achieve scar-less skin removal that has been shown to be safe for the treatment of abdominal and facial skin. Discomfort during microcoring treatment using system 10 is comparable to micro-needling, which is known to be very well tolerated by patients. Further, the healing profile using system 10 is favorable with only transient trace to mild side effects. These clinical results using system 10 demonstrate skin rejuvenation, such as skin tightening and increase in skin thickness after one microcoring treatment using system 10. In some embodiments, multiple microcoring treatments can be performed using system 10, such as to increase efficacy (e.g. improve cosmesis) of the treatment.

Referring now to FIGS. 10A-D, various views of a coring element are illustrated, consistent with the present inventive concepts. Typical dimensions of a coring element 155 are shown.

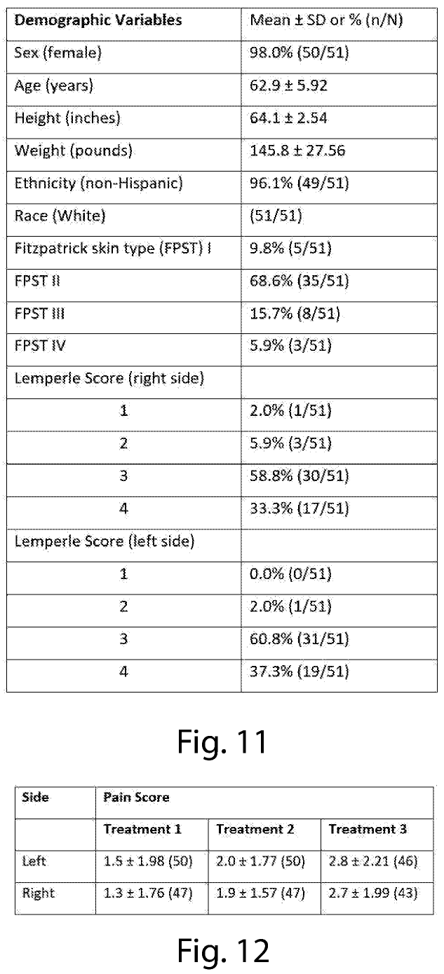

Referring to FIGS. 11-16, applicant has conducted further human clinical studies using the systems, devices, methods, and other technologies of the present inventive concepts, such as system 10 and its components as described herein, to evaluate the safety and efficacy of the treatment of moderate to severe facial wrinkles. Aging skin can be characterized by skin laxity and the appearance of fine lines and wrinkles. Numerous invasive and non-invasive techniques for skin surface area reduction (e.g. skin tightening) are known. Various minimally invasive, nonsurgical treatments such as micro-needling, ablative and non-ablative lasers, radiofrequency, and micro-focused ultrasound have been successfully used to treat mild skin redundancy. Moderate to severe skin redundancy and wrinkling is difficult to improve with these minimally invasive techniques. Facelift surgery provides the most pronounced cosmetic outcomes with respect to the reduction of wrinkles and skin laxity. However, facelift surgery does not address all areas of facial skin laxity (e.g. periorbital and perioral region, nasolabial fold, marionette lines), and is associated with prolonged recovery and the presence of scarring. Microcoring of the present inventive concepts, such as is achieved via use of system 10, combines the benefits of minimally invasive treatment (e.g. fast recovery) with the advantage of scarless skin removal, thereby enabling treatment of moderate to severe skin laxity and wrinkles. Microcoring via system 10, as described herein, uses coring elements 155 comprising hollow coring needles, that when inserted in the skin, excise cores in the size of the needle inner diameter. Compared to micro-needling which only punctures the skin without removing any tissue, microcoring via coring elements 155 and other components of system 10 can remove full-thickness cores of skin, and cores of skin greater than the skin thickness (e.g. greater than 6.0 mm), as well as cores of skin with a thickness between 5.5 mm and 6.0 mm, or cores of skin with a thickness between 5.0 mm and 5.5 mm, or cores of skin with a thickness between 4.5 mm and 5.0 mm, or cores of skin with a thickness between 3.5 mm and 4.0 mm, or cores of skin with a thickness between 2.5 mm and 3.0 mm, or cores of skin with a thickness between 2.0 mm and 2.5 mm, or cores of skin with a thickness between 1.5 mm and 2.0 mm, or cores of skin with a thickness between 1.0 mm and 1.5 mm, or cores of skin with a thickness between 0.5 mm and 1.0 mm, or cores of skin with a thickness less than 0.5 mm. In some embodiments, microcoring via coring elements 155 and other components of system 10 can remove cores of greater than 4.0 mm, such as greater than 5.0 mm, or greater than 6.0 mm, such as when coring element 155 is configured to approach the skin at an angle less than 90 degrees, such as less than 30 degrees, or less than 20 degrees.

Compared to micro-needling which only punctures the skin without removing any tissue, microcoring via coring elements 155 and other components of system 10 can remove cores of skin with diameters less than 1200 microns, such as less than 1000 microns, less than 800 microns, less than 600 microns, less than 400 microns, or less than 300 microns. Additionally, this removal of human skin cores occurs without formation of scars.

Applicant conducted a prospective, multicenter clinical trial that included fifty-one human patients who underwent at least two, and up to three, microcoring treatments via system 10 on both sides of the lower face. Patients underwent study-required visits at baseline (e.g. prior to treatment), treatment (e.g. up to three treatments), and at 1, 7, 30, 60, and 90 days after every treatment. Treatments took place approximately one month apart. Due to the COVID-19 pandemic, some visits could not be completed at the expected time. Amendments were incorporated to allow follow-up visits at 120, 150, or 180 days as the final follow-up. Each side of the face was considered an independent treatment site. Patients who completed at least two treatments (n=51) were included in the final analysis described herein.

The primary endpoint of the study was the Lemperle Wrinkle Severity Scale (LWSS) responder rate. Applicant defined responder as a patient with a reduction of one grade or more on the LWSS at the final follow-up as determined by the investigator. Additionally, an independent expert panel consisting of three reviewers evaluated baseline and post-treatment photographs and determined the LWSS grade (e.g. 0=no wrinkles; 1=just perceptible wrinkles; 2=shallow wrinkles; 3=moderately deep wrinkles; 4=deep wrinkles; well-defined edges; 5=very deep wrinkles, redundant fold). Applicant analyzed the change in the LWSS using repeated measures analysis of variance modeling methods. The model contained a random effect for patients and an effect for side (e.g. left, right). Mean difference from baseline and 95% CI was estimated from the model at a 2-sided 5% alpha-level.

The secondary endpoints of the study were patient satisfaction (e.g. 0=extremely dissatisfied; 1=somewhat dissatisfied; 2=slightly dissatisfied; 3=neither satisfied nor dissatisfied; 4=slightly satisfied; 5=somewhat satisfied; 6=extremely satisfied) and global aesthetic improvement as assessed by the investigator comparing baseline and post-treatment photographs on the GAIS (e.g. 3=very much improved, optimal cosmetic result; 2=much improved, marked improvement in appearance from the initial condition, but not completely optimal; 1=improved, obvious improvement in appearance from the initial condition; 0=no change, the appearance is essentially the same as baseline; −1=worse, the appearance is worse than the original condition; −2=much worse, marked worsening in appearance from the initial condition; −3=very much worse, obvious worsening in appearance from the initial condition).

Treatment endpoints were post-procedure bleeding as assessed by the investigator (e.g. mild, moderate, severe), patient reported pain score (e.g. 0-10), and healing response (e.g. absent, trace, mild, moderate, severe for the following categories: delayed bleeding; hematoma; redness; burning; hyperpigmentation; scarring; crusting; hypopigmentation; skin necrosis; dryness/roughness; infection; skin peeling; ecchymosis; inflammation; tenderness; edema; itching; tightness/pulling; erythema; pain/discomfort; tingling).

As described hereinbelow, applicant has demonstrated a mean LWSS change of 1.3 grades [95% CI: 1.22, 1.42]. Improvement in the GAIS was reported for 89.7% ( 87/97) of treated sites and an average improvement of the GAIS was 1.5. Patients were satisfied with 85.6% ( 83/97) of treatment sites. Post-procedure bleeding and pain was mild with good healing responses by 7 days after treatment. The results demonstrate the safety and efficacy of microcoring treatments (via system 10) for the treatment of moderate to severe facial wrinkles, whereby treatment led to significant improvement of facial wrinkles with high patient satisfaction and fast recovery time. The findings of this trial are summarized hereinbelow.

Referring now to FIG. 11, a table of the baseline demographic variables for the clinical study patients is illustrated, consistent with the present inventive concepts. The study population consisted of predominantly white, non-Hispanic women over 60 years old with Type II or III Fitzpatrick skin type and the LWSS scores of 3 or higher on at least one side (e.g. left, right). A total of 59 patients were screened and enrolled in the study, and subsequently underwent at least one treatment. A total of 53 patients underwent two treatments and a total of 49 patients underwent three treatments. A total of 5 patients (8.5%) discontinued the study or were lost to follow-up before the 90-day follow-up period after the final treatment. Despite the difficulties associated with the COVID-19 pandemic, the overall follow-up visit compliance was 96.1% (571 actual/594 expected visits). Final visits after the final treatment were completed for 54 of the 59 treated patients (91.5%). As shown in FIG. 11, only patients who completed at least two treatments were included in the final analysis (n=51).

Inclusion criteria for the study limited study participants to human patients that were: male or female; having an age between 40-70 years at baseline; having mid to lower face wrinkles with a grade of 3 (e.g. moderately deep wrinkles) and/or 4 (e.g. deep wrinkles, well-defined edges) on at least one side using the LWSS and Fitzpatrick Skin Type I to IV; and able and willing to provide written informed consent and comply with all study related procedures and follow-up visits.

Exclusion criteria for the study excluded study participants that: had lesions suspicious for any malignancy; had the presence of actinic keratosis, melasma, vitiligo, cutaneous papules/nodules, or active inflammatory lesions in the areas to be treated; had a history of keloid formation or hypertrophic scarring; had a history of trauma or surgery in the treatment areas in the past 6 months; had one or more scars present in the areas to be treated; had one or more silicone injections in the areas to be treated, one or more injections of dermal or epidermal fillers in the areas to be treated, fat or botulinum toxin in the areas to be treated, or had undergone any other facial procedure, within the study treatment areas, within the past 6 months (e.g. dermabrasion, laser, RF, chemical and mechanical peels); had an active smoking status or have quit within 3 months before treatment; had an active, chronic, or recurrent infection; had a history of compromised immune system or currently being treated with immunosuppressive agents; had a history of sensitivity or allergy to any topical, injectable, or other preparation used during the study such as Aquaphor®, topical or injected anesthetics (e.g. lidocaine, benzocaine, procaine, chlorhexidine, povidone-iodine, or epinephrine); had excessive sun exposure, a use of tanning beds, or use of tanning creams within 30 days before treatment and for the duration of the study; had undergone treatment with aspirin or other blood thinning agents within 14 days before treatment; had a history or presence of any clinically significant bleeding disorder; had a treatment with an investigational device or agent within 30 days before treatment or during the study period; and/or were currently pregnant or breast-feeding, or planning to become pregnant during the study period.

Referring now to FIG. 12, a table of the mean post-procedure pain scores as reported by the clinical study patients is illustrated, consistent with the present inventive concepts. Applicant provided a pain score scale of 0-10 to be associated with each treatment, whereby a pain score of 0 indicates no pain and a pain score of 10 indicates worst pain possible. As shown in FIG. 12, patients reported mean post-procedure pain scores of between 1.2-2.8. In comparison, patients undergoing micro-needling treatment have been shown to experience pain relating to a pain score of 0.2-3.8 out of 10 during treatment. System 10 can provide a microcoring treatment with similar, or slightly less, discomfort during the microcoring treatment as compared to micro-needling procedures. System 10 can provide a microcoring treatment of the present inventive concepts that results in a pain score of no more than 3.5, or no more than 3.0.

The microcoring treatments performed using system 10 were performed using coring elements 155 each comprising a 22-gauge needle, and coring densities of 6.5%, 6.7%, 7.9%, and/or 8.5% (percent of skin removed per 1 cm²). Coring depths were between 3 mm and 4 mm. The minimum core count was 6,000 microcores. Treatment location was limited to the mid to lower face. Upon completion of the treatment, the area was rinsed with sterile saline and Aquaphor® was applied. Patients were instructed to apply Aquaphor® daily until wound healing occurred.

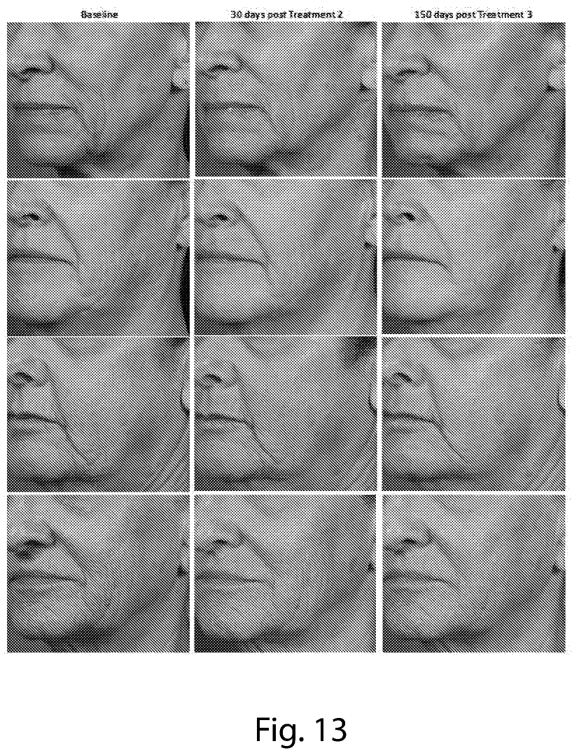

Referring now to FIG. 13, a series of representative patient photographs taken before and after treatment are illustrated, consistent with the present inventive concepts. Patient photographs were taken at baseline (e.g. before treatment), 30 days after a second treatment, and 150 days after a third treatment. It was found the Lemperle Wrinkle Severity Scale (LWSS) improved by greater than or equal to 1 grade in 82.8% [95% CI; 77.98%-86.97%] of the treatment areas. The mean change from baseline for the LWSS was 1.3 grade [95% CI. 1.22, 1.42]. Additionally, an independent reviewer panel were able to correctly identify 92.1% (268/291) of the 90-day post-treatment photos as post-treatment. System 10 can be configured to perform a microcoring procedure on a patient that results in an LWSS improvement of at least 0.1, such as at least 0.2, 0.3 and/or 0.5, such as when the improvement is achieved in at least 50%, 75% and/or 85% of the patients treated (e.g. each patient has a 50%, 75%, and/or 85% likelihood of achieving the LWSS improvement).

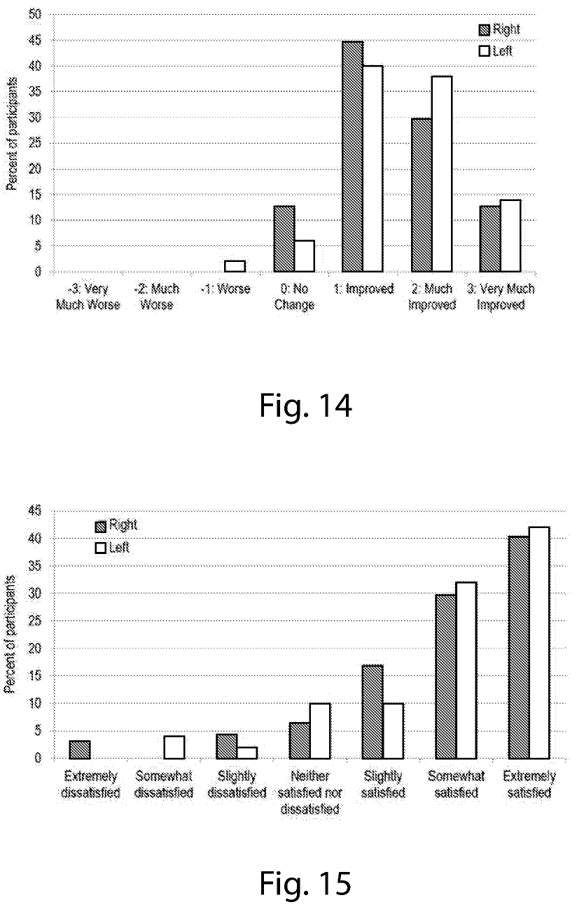

Referring now to FIG. 14, a graph demonstrating the global aesthetic improvement scale (GAIS) change before and after treatment by side is illustrated, consistent with the present inventive concepts. Improvement in the GAIS was reported for 89.7% ( 87/97) of treated sides. The mean change in the GAIS indicates an improved score of 1.5 at the last treatment follow-up compared to baseline (e.g. before treatment). System 10 can be configured to perform a microcoring procedure on a patient that results in a GAIS improvement, such as an improvement of at least 1.0, such as when the improvement is achieved in at least 50%, 75% and/or 85% of the patients treated (e.g. each patient has a 50%, 75%, and/or 85% likelihood of achieving the GAIS improvement).

Referring now to FIG. 15, a graph demonstrating patient satisfaction after treatment by side is illustrated, consistent with the present inventive concepts. When considering all treatment sides, the overall satisfaction rate (e.g. slightly, somewhat, and extremely satisfied) was 85.6% ( 83/97). System 10 can be configured to perform a microcoring procedure on a patient that results in a significant level of patient satisfaction, such as when at least 50%, 75% and/or 85% of the patients treated are sufficiently satisfied (e.g. each patient has a 50%, 75%, and/or 85% likelihood of being sufficiently satisfied).

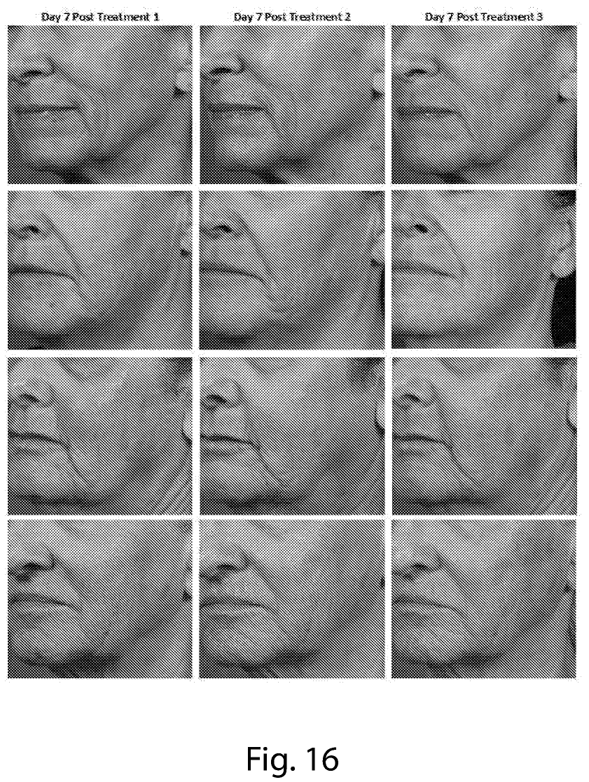

Referring now to FIG. 16, a series of representative patient photographs taken after treatment are illustrated, consistent with the present inventive concepts. Patient photographs were taken 7 days after the first treatment, 7 days after a second treatment, and 7 days after a third treatment. Applicant found that 90% of patients had absent, trace, or mild healing responses by 7 days after treatment. Post-procedure bleeding was mild in most cases (greater than or equal to 78%). There were no reports of severe bleeding by the patients. System 10 can be configured to perform a microcoring procedure on a patient that results in absent, trace, and/or mild healing responses by 7 days after treatment. System 10 can be configured to perform a microcoring procedure in which at least 50%, 60%, and/or 75% of the patients treated have no more than mild bleeding post-procedure (e.g. each patient has a 50%, 60%, and/or 75% likelihood of having no more than mild bleeding post-procedure).

Moderate responses to ecchymosis, edema, erythema, hyperpigmentation, itching, pain and/or discomfort, redness, tenderness, and tightness were seen in less than 10% of cases at 7 days. A limited number (less than 5%) of patients reported moderate responses to dryness, ecchymosis, erythema, hyperpigmentation, and redness at 30 days after treatment. No skin reactions were reported at 90 days after treatment. System 10 can be configured to perform a microcoring procedure on a patient that avoids significant responses related to ecchymosis, edema, erythema, hyperpigmentation, itching, pain and/or discomfort, redness, tenderness, and/or tightness, such as when at least 50%, 75% and/or 85% of the patients treated avoid significant responses to these undesired conditions (e.g. each patient has a 50%, 75%, and/or 85% likelihood of avoiding these undesired conditions).

Additionally, no serious adverse events were reported. A total of nine adverse events were reported in five patients. Five adverse events were not related to the device or treatment. Four of the adverse events in four patients were considered adverse device effects (ADEs), which included black eye, cheek numbness, redness, and prolonged needle marks on cheek. The ADEs were mild to moderate in severity and did not require any intervention. Applicant found that all adverse events were resolved by the end of the study.

As described herein, applicant has demonstrated treatment of facial skin via system 10 is well tolerated with minimal pain and bleeding during treatment, as well as short recovery time and good healing profile. Additionally, treatment via system 10 can lead to significant improvement of moderate to severe facial wrinkles with high patient satisfaction.

Applicant found treatment improved lower face wrinkles by greater than or equal to 1 grade on the LWSS in 83% of patients with a mean change of 1.3 at 90 days after treatment. In comparison, four sessions of micro-needling 30 days apart have been shown to lead to a mean change in wrinkle severity of 0.4 for nasolabial folds, and 0.3 for marionette lines at 90 days after treatment. Therefore, treatment via system 10 appears to be three times as effective as micro-needling for the reduction of wrinkles. This change in the LWSS was further reflected by the improvements seen in overall aesthetic appearance of the lower face on the GAIS in the vast majority (89.7%) of treated sides. Patients were satisfied with the treatment outcome in most cases (85.6%).

As demonstrated in prior safety studies, treatment via system 10 is well tolerated with standard pain management. Bleeding was mild in most cases and the mean post-procedure pain scores were between 1.2-2.8 (on a scale of 0-10). In comparison, patients undergoing micro-needling treatment using system 10 have been shown to experience a pain level of 0.2-3.8 out of 10 during treatment.

Comparable to prior clinical trials using system 10 (e.g. as described herein), the system 10 healing profile was favorable with most patients (approximately 78%) experiencing full recovery after treatment at 7 days. The most common skin reactions (e.g. erythema, edema, ecchymosis, redness, itching, tightness) were equivalent to those seen after micro-needling procedures. Although skin reactions were comparable to fractional CO₂ laser treatments, complications that commonly occur after laser treatment, such as bacterial and viral infections, were not observed. The reported skin reactions were trace to mild, and no moderate or severe skin responses to treatment were observed. The skin recovery time from treatment via system 10 is slightly longer than for micro-needling procedures, and significantly shorter than for fractional CO₂ laser resurfacing, after which most patients experience side effects for approximately 14 days.

No long-term skin reactions were observed at the last follow-up visit, including a lack of hypopigmentation and hyperpigmentation. System 10 can be void of the use of laser and/or other energies that generate heat in the skin, therefore reducing the risk of pigment disturbances (e.g. post-inflammatory hyperpigmentation and/or hypopigmentation) as compared to thermally ablative devices that are often associated with pigment disturbances.

The results of these human clinical studies using the microcoring-based treatment of system 10 demonstrate the safety and efficacy of system 10 for the reduction of moderate to severe facial wrinkles. Treatment via system 10 leads to significant improvement of facial wrinkles with high patient satisfaction and fast recovery time. System 10 provides a safe and effective minimally invasive treatment of wrinkles of the face.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A system for producing a cosmetic effect in skin tissue of a patient, the system comprising:
a treatment module comprising at least one coring element configured to remove a portion of skin tissue when the coring element is inserted into and withdrawn from the skin tissue; and
an actuation assembly operably attached to the treatment module and configured to translate and/or actuate the treatment module in one or more directions relative to a surface of the skin tissue;
wherein the system is configured to perform a microcoring procedure that provides a cosmetic effect to the patient.

2.-57. (canceled)
